(19) United States
(12) Patent Application Publication
(10) Pub. No.: US 2023/0113775 A1
(43) Pub. Date: Apr. 13, 2023

(54) ANHYDROUS COMPOSITIONS OF EGFR INHIBITORS AND METHODS OF USE

(71) Applicant: PALVELLA THERAPEUTICS, INC., Wayne, PA (US)

(72) Inventors: Braham SHROOT, Antibes (FR); Jeffrey MARTINI, Malvern, PA (US); Wesley Harton KAUPINEN, Wayne, PA (US)

(73) Assignee: PALVELLA THERAPEUTICS, INC., Wayne, PA (US)

(21) Appl. No.: 17/930,570

(22) Filed: Sep. 8, 2022

Related U.S. Application Data
(60) Provisional application No. 63/364,842, filed on May 17, 2022.
(60) Provisional application No. 63/274,629, filed on Nov. 2, 2021.
(60) Provisional application No. 63/242,702, filed on Sep. 10, 2021.

(51) Int. Cl. A61K 31/517; A61K 45/06; A61K 47/38; A61P 17/00

(52) U.S. Cl. CPC A61K 31/517; A61K 45/06; A61K 47/38; A61P 17/00

(57) ABSTRACT

Disclosed herein are compositions and methods for topical delivery of EGFR inhibitors. In one embodiment, a topical anhydrous composition includes one or more EGFR inhibitors, one or more solvents, one or more gelling agents, and, optionally, one or more antioxidants. Also disclosed herein are methods to treat skin disorders using such compositions.

### CROSS REFERENCE TO RELATED APPLICATIONS

This application claims priority to U.S. Provisional Application No. 63/364,842 filed May 17, 2022, U.S. Provisional Application No. 63/274,629 filed Nov. 2, 2021, and U.S. Provisional Application No. 63/242,702 filed Sep. 10, 2021, which are hereby incorporated by reference in their entirety.

### SUMMARY OF THE INVENTION

Disclosed herein are compositions and methods for topical delivery of EGFR inhibitors. In one embodiment, a topical anhydrous composition comprises an effective amount of one or more EGFR inhibitors, one or more solvents, and one or more gelling agents. In some embodiments, the EGFR inhibitor is present from about 0.1 wt % to about 20 wt % of the topical anhydrous composition. In some embodiments, the solvent is present from about 75 wt % to about 99.9 wt % of the topical anhydrous composition. In some embodiments, the gelling agent is present from about 0.1 wt % to about 5 wt % of the topical anhydrous composition.

Disclosed herein are compositions and methods for topical delivery of EGFR inhibitors. In one embodiment, a topical anhydrous composition comprises an effective amount of one or more EGFR inhibitors, one or more solvents, one or more gelling agents, and one or more antioxidants. In some embodiments, the EGFR inhibitor is present from about 0.1 wt % to about 20 wt % of the topical anhydrous composition. In some embodiments, the solvent is present from about 75 wt % to about 99.9 wt % of the topical anhydrous composition. In some embodiments, the gelling agent is present from about 0.1 wt % to about 5 wt % of the topical anhydrous composition. In some embodiments, the antioxidant is present from about 0.001 wt % to about 1 wt % of the topical anhydrous composition.

In additional embodiments, methods of treating a skin disorder in a subject in need thereof comprises topically administering an effective amount of a topical anhydrous composition comprising an effective amount of one or more EGFR inhibitors, one or more solvents, and one or more gelling agents. In some embodiments, the EGFR inhibitor is present from about 0.1 wt % to about 20 wt % of the topical anhydrous composition. In some embodiments, the solvent is present from about 75 wt % to about 99.9 wt % of the topical anhydrous composition. In some embodiments, the gelling agent is present from about 0.1 wt % to about 5 wt % of the topical anhydrous composition.

In additional embodiments, methods of treating a skin disorder in a subject in need thereof comprises topically administering an effective amount of a topical anhydrous composition comprising an effective amount of one or more EGFR inhibitors, one or more solvents, one or more gelling agents, and one or more antioxidants. In some embodiments, the EGFR inhibitor is present from about 0.1 wt % to about 20 wt % of the topical anhydrous composition. In some embodiments, the solvent is present from about 75 wt % to about 99.9 wt % of the topical anhydrous composition. In some embodiments, the gelling agent is present from about 0.1 wt % to about 5 wt % of the topical anhydrous composition. In some embodiments, the antioxidant is present from about 0.001 wt % to about 1 wt % of the topical anhydrous composition.

### BRIEF DESCRIPTION OF THE DRAWINGS

FIGS. 1A-B shows the effect of erlotinib on keratinocytes. FIG. 1A. Graph showing decreased keratinocyte proliferation with medium and high doses of erlotinib. FIG. 1B. shows keratinocytes treated with vehicle and 100 nM, 500 nM, and 1000 nM doses of erlotinib.

FIG. 2 shows the effect of erlotinib on non-diseased 3D human skin equivalent (HSE).

FIGS. 3A-B show the effect of erlotinib on non-diseased donor tissue and epidermolytic ichthyosis (EI) donor tissue. FIG. 3A. shows keratinocytes derived from non-diseased donor tissue and keratinocytes derived from EI donor tissue treated with vehicle or 100 nM dose erlotinib. A. Microscopic images of non-diseased and EI derived keratinocytes following exposure to 100 nM erlotinib showing a marked reduction in colony density. FIG. 3B. Graph showing fold change in EI derived keratinocyte colony density following exposure to 100 nM erlotinib.

FIGS. 4A-B show the effect of erlotinib on 3D non-diseased HSE and 3D EI HSE. FIG. 4A. shows non-diseased 3D HSE treated with vehicle or 100 nM dose erlotinib. FIG. 4B. shows 3D EI HSE treated with vehicle or 100 nM erlotinib. A marked morphologic change is produced in the upper epidermis in erlotinib treated EI constructs suggesting that aberrant proliferation and differentiation in the disease can be decreased by this agent.

FIGS. 5A-B shows TEER analysis of 3D non-diseased HSE and 3D EI HSE after treatment with erlotinib. FIG. 5A. non-diseased 3D HSE treated with 10 nM and 100 nM erlotinib. FIG. 5B. EI 3D HSE treated with 10 nM and 100 nM erlotinib. Erlotinib treatment did not produced a significant change in TEER values compared to controls, implying that skin barrier function is not compromised by erlotinib treatment.

FIGS. 6A-B shows the effect of erlotinib in a mouse model of hyperkeratosis. FIG. 6A. Western blot analysis of protein expression of p^{Y1068}EGFR, EGFR, KRT10 and KRT16 in mice treated with vehicle, vehicle+TPA, and TPA+three formulations of erlotinib at 0.4%, 1% and 4% strength. FIG. 6B. Fold increase in expression of pY1068EGFR, EGFR, KRT10 and KRT16 in mice treated with vehicle, vehicle+TPA, and TPA+increasing doses of erlotinib.

### DETAILED DESCRIPTION OF THE INVENTION

Where a range of values is provided, it is intended that each intervening value between the upper and lower limit of that range and any other stated or intervening value in that stated range is encompassed within the disclosure. For example, if a range of 1 μm to 8 μm is stated, it is intended that 2 μm, 3 μm, 4 μm, 5 82 m, 6 μm, and 7 μm are also explicitly disclosed, as well as the range of values greater than or equal to 1 μm and the range of values less than or equal to 8 μm.

As used in this application and in the claims, the singular forms “a,” “an,” and “the” include the plural forms unless the context clearly dictates otherwise. Additionally, the term “includes” means “comprises.”

As used herein, all claimed numeric terms are to be read as being preceded by the term, “about,” which means plus or minus 10% of the numerical value of the number with which it is being used. Therefore, a claim to darier “50%” means “about 50%” and encompasses the range of 45%-55%.

The term “patient” and “subject” are interchangeable and may be taken to mean any living organism which may be treated with compounds of the present invention. As such, the terms “patient” and “subject” may include, but is not limited to, any non-human mammal, primate or human. In some embodiments, the “patient” or “subject” is a mammal, such as mice, rats, other rodents, rabbits, dogs, cats, swine, cattle, sheep, horses, primates, or humans. In some embodiments, the patient or subject is an adult, child or infant. In some embodiments, the patient or subject is a human.

“Administering” when used in conjunction with the EGFR inhibitor means to administer EGFR inhibitor to a patient whereby the EGFR inhibitor positively impacts the tissue to which it is targeted. The EGFR inhibitors described herein can be administered either alone or in combination (concurrently or serially) with other pharmaceutically active agents. For example, the EGFR inhibitors can be administered in combination with other anti-cancer or anti-neoplastic agents, or in combination with other therapies for treating skin disorders. In some embodiments, the EGFR inhibitors described herein can also be administered in combination with (i.e., as a combined composition or as separate compositions) other therapeutics.

An “effective amount” of a composition is a predetermined amount calculated to achieve the desired effect, i.e., to ameliorate, prevent or improve an unwanted condition, disease or symptom of a patient. The activity contemplated by the present methods may include both therapeutic and/or prophylactic treatment, as appropriate. The specific dose of the agent administered according to this invention to obtain therapeutic and/or prophylactic effects will, of course, be determined by the particular circumstances surrounding the case, including, for example, the compound administered, the route of administration, and the condition being treated. The effective amount administered may be determined by a physician in the light of the relevant circumstances including the condition to be treated, the choice of the compound to be administered, and the chosen route of administration.

The term “carrier” as used herein encompasses carriers, excipients, and diluents, meaning a material, composition or vehicle, such as a liquid or solid filler, diluent, excipient, solvent or encapsulating material involved in carrying or transporting a pharmaceutical, cosmetic or other agent across a tissue layer such as the stratum corneum or stratum spinosum.

The transitional term “comprising,” which is synonymous with “including,” “containing,” or “characterized by,” is inclusive or open-ended and does not exclude additional, unrecited elements or method steps. By contrast, the transitional phrase “consisting of excludes any element, step, or ingredient not specified in the claim. The transitional phrase “consisting essentially of’ limits the scope of a claim to the specified materials or steps “and those that do not materially affect the basic and novel characteristic(s)” of the claimed invention. In embodiments or claims where the term comprising is used as the transition phrase, such embodiments can also be envisioned with replacement of the term “comprising” with the terms “consisting of’ or “consisting essentially of.”

The term “treating” is used herein, for instance, in reference to methods of treating a skin disorder or a systemic condition, and generally includes the administration of a compound or composition which reduces the frequency of, or delays the onset of, symptoms of a medical condition or enhance the texture, appearance, color, sensation, or hydration of the intended tissue treatment area of the tissue surface in a subject relative to a subject not receiving the compound or composition. This can include reversing, reducing, or arresting the symptoms, clinical signs, and underlying pathology of a condition in a manner to improve or stabilize a subject's condition.

The term “disorder” is used in this disclosure to mean, and is used interchangeably with, the terms disease, condition, or illness, unless otherwise indicated.

The weight percentages disclosed herein are weight-to-weight or weight-to-volume percentages, as appropriate.

Disclosed herein are anhydrous compositions of EGFR inhibitors. In some embodiments, the topical anhydrous compositions comprise one or more EGFR inhibitors, one or more solvents, and one or more gelling agents.

Disclosed herein are anhydrous compositions of EGFR inhibitors. In some embodiments, the topical anhydrous compositions comprise one or more EGFR inhibitors, one or more solvents, one or more gelling agents, and one or more antioxidants.

In some embodiments, the topical anhydrous composition of EGFR inhibitor has a Cₘₐₓ of about 120-990 micromolar in the epidermis, and about 36-350 micromolar in the dermis. In some embodiments, the topical anhydrous composition of EGFR inhibitor has a Tₘₐₓ of about 15-24 hours in the epidermis. In some embodiments, the topical anhydrous composition is considered to be bioequivalent or substantially bioequivalent, as measured by accepted topical bioavailability studies, to a topical anhydrous composition comprising an EGFR inhibitor as described herein.

In some embodiments, the topical anhydrous composition comprises at least one EGFR inhibitor. The at least one EGFR inhibitors is selected from erlotinib, osimertinib (AZD9291), neratinib, gefitinib, dacomitinib, lapatinib, vandetanib, afatinib, icotinib, rociletinib, naquotinib (ASP8273), and pharmaceutically acceptable salts, hydrates, solvates, polymorphs, or amorphous solids thereof. In preferred embodiments, the topical anhydrous composition comprises erlotinib free base.

In some embodiments, the topical anhydrous composition may further comprise other compounds regulating EGFR, such as monoclonal antibodies selected from panitumumab, nimotuzumab, necitumumab, LY3016859, or cetuximab.

In some embodiments, the at least one EGFR inhibitor is present from about 0.1 wt % to about 20 wt % of the topical anhydrous composition, about 0.1 wt % to about 15 wt % of the topical anhydrous composition, about 0.1 wt % to about 10 wt % of the topical anhydrous composition, about 0.1 wt % to about 6 wt % of the topical anhydrous composition, about 0.1 wt % to about 5 wt % of the topical anhydrous composition, or about 0.1 wt % to about 4 wt % of the topical anhydrous composition, and any individual amount or any ranges between any two of these values. Non-limiting examples include, about 0.1 wt %, about 0.2 wt %, about 0.3 wt %, about 0.4 wt %, about 0.5 wt %, about 0.6 wt %, about 0.7 wt %, about 0.8 wt %, about 0.9 wt %, about 1 wt %, about 1.5 wt %, about 2 wt %, about 2.5 wt %, about 3 wt %, about 3.5 wt %, about 4 wt %, about 4.5 wt %, about 5 wt %, about 6 wt %, about 7 wt %, about 8 wt %, about 9 wt %, about 10 wt %, about 15 wt %, or about 20 wt %. In some embodiments, the EGFR inhibitor is erlotinib free base and is present from about 0.1 wt % to about 7 wt % of the topical anhydrous composition. Preferably, the at least one EGFR inhibitor is erlotinib free base and is present at about 0.3 wt %, about 1 wt %, about 3 wt %, or about 4 wt %.

In some embodiments, the compound regulating EGFR is present from about 0.1 wt % to about 20 wt % of the topical anhydrous composition, about 0.1 wt % to about 15 wt % of the topical anhydrous composition, about 0.1 wt % to about 10 wt % of the topical anhydrous composition, about 0.1 wt % to about 6 wt % of the topical anhydrous composition, about 0.1 wt % to about 5 wt % of the topical anhydrous composition, or about 0.1 wt % to about 4 wt % of the topical anhydrous composition, and any individual amount or any ranges between any two of these values. Non-limiting examples include, about 0.1 wt %, about 0.2 wt %, about 0.3 wt %, about 0.4 wt %, about 0.5 wt %, about 0.6 wt %, about 0.7 wt %, about 0.8 wt %, about 0.9 wt %, about 1 wt %, about 1.5 wt %, about 2 wt %, about 2.5 wt %, about 3 wt %, about 3.5 wt %, about 4 wt %, about 4.5 wt %, about 5 wt %, about 6 wt %, about 7 wt %, about 8 wt %, about 9 wt %, about 10 wt %, about 15 wt %, or about 20 wt %.

In some embodiments, the one or more solvents is selected from alcohols, polyols, amides, esters, propylene glycol ethers, or mixtures thereof.

In some embodiments, the alcohol or polyol is selected from ethanol, isopropanol, butanol, benzyl alcohol, ethylene glycol, propylene glycol, propylene glycol monocaprylate, diglycol, diethylene glycol monoethylether, tetrahydrofurfurylalcohol polyethylene glycol ether (glycofurol), butylene glycol, diethylene glycol, triethylene glycol, PEG 400, PEG 3350, SR-PEG 400, SR-DMI, oeyl alcohol, castor oil, miglyol 810, liquid paraffin, propylene glycol dicaprylate/dicaprate, butanediols, isomers of butanediols, glycerol (AKA glycerin), glycerol triacetate, pentaerythritol, sorbitol, mannitol, diisopropyl adipate, dimethyl isosorbide, polyethylene glycol, polypropylene glycol, polyvinylalcohol, hydroxypropyl methylcellulose, cellulose derivatives, cyclodextrins and cyclodextrin derivatives, or mixtures thereof.

In some embodiments, the amide is selected from 2-pyrrolidone, 2-piperidone, c-caprolactam, N-alkylpyrrolidone, N-hydroxyalkylpyrrolidone, N-alkylpiperidone, N-alkylcaprolactam, dimethylacetamide, polyvinylpyrrolidone, or mixtures thereof.

In some embodiments, the ester is selected from ethyl propionate, tributylcitrate, acetyl triethylcitrate, acetyl tributyl citrate, triethylcitrate, ethyl oleate, ethyl caprylate, ethyl butyrate, triacetin, propylene glycol monoacetate, propylene glycol diacetate, c-caprolactone, isomers of c-caprolactone, 6-valerolactone, isomers of 6-valerolactone, P-butyrolactone, isomers of P-butyrolactone, or mixtures thereof.

Preferably, one or more solvents are selected from PEG400, diisopropyl adipate (DIA), glycerol (AKA glycerin), isopropanol (IPA), or mixtures thereof.

In some embodiments, the topical anhydrous composition does not contain ethanol. In some embodiments, the topical anhydrous composition contains benzyl alcohol at less than 5 wt %, less than 4 wt %, less than 3 wt %, less than 2 wt %, less than 1.5 wt %, or less than 1 wt %. In some embodiments, the topical anhydrous composition does not contain benzyl alcohol.

In some embodiments, the total amount of the one or more solvent is present from about 75 wt % to about 99.9 wt % of the topical anhydrous composition, about 80 wt % to about 99.9 wt % of the topical anhydrous composition, about 85 wt % to about 99.9 wt % of the topical anhydrous composition, about 90 wt % to about 99.9 wt % of the topical anhydrous composition, about 95 wt % to about 99.9 wt % of the topical anhydrous composition, about 75 wt % to about 95 wt % of the topical anhydrous composition, about 75 wt % to about 90 wt % of the topical anhydrous composition, about 75 wt % to about 85 wt % of the topical anhydrous composition, or about 75 wt % to about 80 wt % of the topical anhydrous composition. Non-limiting examples include, about 75 wt %, about 80 wt %, about 85 wt %, about 90 wt %, about 91 wt %, about 92 wt %, about 93 wt %, about 94 wt %, about 95 wt %, about 96 wt %, about 97 wt %, about 98 wt %, about 99 wt %, or about 99.9 wt %. Preferably, the total solvent is present from about 85 wt % to about 95 wt %.

In some embodiments, each solvent is present from about 10 wt % to about 60 wt % of the topical anhydrous composition, about 10 wt % to about 50 wt % of the topical anhydrous composition, about 10 wt % to about 40 wt % of the topical anhydrous composition, about 10 wt % to about 30 wt % of the topical anhydrous composition, about 10 wt % to about 20 wt % of the topical anhydrous composition, about 10 wt % to about 15 wt % of the topical anhydrous composition, about 20 wt % to about 60 wt % of the topical anhydrous composition, about 30 wt % to about 60 wt % of the topical anhydrous composition, about 35 wt % to about 60 wt % of the topical anhydrous composition, about 40 wt % to about 60 wt % of the topical anhydrous composition, about 45 wt % to about 60 wt % of the topical anhydrous composition, about 50 wt % to about 60 wt % of the topical anhydrous composition, or about 55 wt % to about 60 wt % of the topical anhydrous composition.

In some embodiments, the one or more gelling agents is selected from poloxamers, carbomers, or mixtures thereof.

In some embodiments, the poloxamer is selected from poloxamer P-188, poloxamer P-138, poloxamer P-237, poloxamer P-288, poloxamer P-124, poloxamer P338, poloxamer P-407, poly(ethylene glycol/DL-lactide-Co-glyceride) poly(caprolactum), hydroxypropyl cellulose (KLUCEL®), glyceryl tris 12-hydroxy stearate, hydroxy stearin, propylene carbonate, polyvinyl pyrolidine, or mixtures thereof.

In some embodiments, the carbomers is selected from carbomer 981, carbomer 934, carbomer 934P, carbomer 940, carbomer 941, carbomer 1342, polycarbophil, calcium polycarbophil, or mixtures thereof.

Preferably, the one or more gelling agent is hydroxypropyl cellulose (HPC).

In some embodiments, the one or more gelling agent is present from about 0.1 wt % to about 6 wt % of the topical anhydrous composition, about 0.1 wt % to about 5 wt % of the topical anhydrous composition, about 0.1 wt % to about 4 wt % of the topical anhydrous composition, about 0.1 wt % to about 3 wt % of the topical anhydrous composition, about 0.1 wt % to about 2 wt % of the topical anhydrous composition, or about 0.1 wt % to about 1 wt % of the topical anhydrous composition. Preferably, the gelling agent is present from about 0.5 wt % to about 1 wt %. Most preferably, the gelling agent is present at about 0.75 wt %.

In some embodiments, the one or more antioxidants is selected from ascorbic acid, vitamin E and its derivatives, a-tocopherol, iv-tocopherol, 5-tocopherol, ascorbyl palmitate, propyl gallate (PG), octyl gallate, dodecyl gallate, butylated hydroxy anisole (BHA), butylated hydroxy toluene (BHT), D-a-tocopheryl polyethylene glycol 1000 succinate, or mixtures thereof. Preferably, the one or more antioxidants is selected from propyl gallate, ascorbyl palmitate, a-tocopherol, or mixtures thereof.

In some embodiments, the one or more antioxidant is present from about 0.001 wt % to about 1 wt % of the topical anhydrous composition, about 0.001 wt % to about 0.1 wt % of the topical anhydrous composition, about 0.001 wt % to about 0.01 wt % of the topical anhydrous composition, or about 0.01 wt % to about 0.1 wt % of the topical anhydrous composition. Preferably, the antioxidant is present at about 0.072% of the topical anhydrous composition.

In some embodiments, the topical anhydrous composition comprises an effective amount of one or more EGFR inhibitors present from about 0.1 wt % to about 20 wt % of the topical anhydrous composition, one or more solvents present from about 75 wt % to about 99.9 wt % of the topical anhydrous composition, and one or more gelling agents present from about 0.1 wt % to about 6 wt % of the topical anhydrous composition.

In some embodiments, the topical anhydrous composition comprises an effective amount of one or more EGFR inhibitors present from about 0.1 wt % to about 20 wt % of the topical anhydrous composition, one or more solvents present from about 75 wt % to about 99.9 wt % of the topical anhydrous composition, one or more gelling agents present from about 0.1 wt % to about 6 wt % of the topical anhydrous composition, and one or more antioxidants present from about 0.001 wt % to about 1 wt % of the topical anhydrous composition.

In some embodiments, the topical anhydrous composition comprises an effective amount of one or more EGFR inhibitors present from about 0.1 wt % to about 10 wt % of the topical anhydrous composition, one or more solvents present from about 85 wt % to about 95 wt % of the topical anhydrous composition, and one or more gelling agents present from about 0.1 wt % to about 1 wt % of the topical anhydrous composition.

In some embodiments, the topical anhydrous composition comprises an effective amount of one or more EGFR inhibitors present from about 0.1 wt % to about 10 wt % of the topical anhydrous composition, one or more solvents present from about 85 wt % to about 95 wt % of the topical anhydrous composition, one or more gelling agents present from about 0.1 wt % to about 1 wt % of the topical anhydrous composition, and one or more antioxidants present from about 0.01 wt % to about 1 wt %. of the topical anhydrous composition.

In some embodiments, the topical anhydrous composition comprises an effective amount of one or more EGFR inhibitors present from about 1 wt % to about 10 wt % of the topical anhydrous composition, one or more solvents present from about 85 wt % to about 95 wt % of the topical anhydrous composition, and one or more gelling agents present from about 0.1 wt % to about 1 wt % of the topical anhydrous composition.

In some embodiments, the topical anhydrous composition comprises an effective amount of one or more EGFR inhibitors present from about 1 wt % to about 10 wt % of the topical anhydrous composition, one or more solvents present from about 85 wt % to about 95 wt % of the topical anhydrous composition, one or more gelling agents present from about 0.1 wt % to about 1 wt % of the topical anhydrous composition, and one or more antioxidants present from about 0.01 wt % to about 1 wt %. of the topical anhydrous composition.

In some embodiments, the topical anhydrous composition comprises an effective amount of one or more EGFR inhibitors present from about 1 wt % to about 5 wt % of the topical anhydrous composition, one or more solvents present from about 85 wt % to about 95 wt % of the topical anhydrous composition, and one or more gelling agents present from about 0.1 wt % to about 1 wt % of the topical anhydrous composition.

In some embodiments, the topical anhydrous composition comprises an effective amount of one or more EGFR inhibitors present from about 1 wt % to about 5 wt % of the topical anhydrous composition, one or more solvents present from about 85 wt % to about 95 wt % of the topical anhydrous composition, one or more gelling agents present from about 0.1 wt % to about 1 wt % of the topical anhydrous composition, and one or more antioxidants present from about 0.01 wt % to about 1 wt %. of the topical anhydrous composition.

In some embodiments, the topical anhydrous composition comprises an effective amount of one or more EGFR inhibitors present from about 0.1 wt % to about 4.5 wt % of the topical anhydrous composition, one or more solvents present from about 80 wt % to about 99.9 wt % of the topical anhydrous composition, and one or more gelling agents present from about 0.1 wt % to about 4 wt % of the topical anhydrous composition.

In some embodiments, the topical anhydrous composition comprises an effective amount of one or more EGFR inhibitors present from about 0.1 wt % to about 4.5 wt % of the topical anhydrous composition, one or more solvents present from about 80 wt % to about 99.9 wt % of the topical anhydrous composition, one or more gelling agents present from about 0.1 wt % to about 4 wt % of the topical anhydrous composition, and one or more antioxidants present from about 0.01 wt % to about 1 wt %. of the topical anhydrous composition.

In some embodiments, the topical anhydrous composition comprises an effective amount of one or more EGFR inhibitors present at about 3.9 wt % of the topical anhydrous composition, one or more solvents present from about 90 wt % to about 96 wt % of the topical anhydrous composition, and one or more gelling agents present from about 0.01 wt % to about 5.75 wt % of the topical anhydrous composition.

In some embodiments, the topical anhydrous composition comprises an effective amount of one or more EGFR inhibitors present at about 3.9 wt % of the topical anhydrous composition, one or more solvents present from about 90 wt % to about 96 wt % of the topical anhydrous composition, one or more gelling agents present from about 0.01 wt % to about 5.75 wt % of the topical anhydrous composition, and one or more antioxidants present from about 0.001 wt % to about 5 wt %. of the topical anhydrous composition.

In some embodiments, the topical anhydrous composition comprises erlotinib at about 0.3 wt % of the topical anhydrous composition, isopropyl alcohol is present at about 20 wt % of the topical anhydrous composition, PEG400 is present at about 40.25 wt % of the topical anhydrous composition, diisopropyl adipate is present at about 20 wt % of the topical anhydrous composition, glycerol is present at about 20 wt % of the topical anhydrous composition, and hydroxypropyl cellulose is present at about 0.75 wt % of the topical anhydrous composition.

In some embodiments, the topical anhydrous composition comprises erlotinib at about 1 wt % of the topical anhydrous composition, isopropyl alcohol is present at about 18 wt % of the topical anhydrous composition, PEG400 is present at about 43.25 wt % of the topical anhydrous composition, diisopropyl adipate is present at about 20 wt % of the topical anhydrous composition, glycerol is present at about 20 wt % of the topical anhydrous composition, and hydroxypropyl cellulose is present at about 0.75 wt % of the topical anhydrous composition.

In some embodiments, the topical anhydrous composition comprises erlotinib at about 4 wt % of the topical anhydrous composition, isopropyl alcohol is present at about 15 wt % of the topical anhydrous composition, PEG400 is present at about 40.25 wt % of the topical anhydrous composition, diisopropyl adipate is present at about 20 wt % of the topical anhydrous composition, glycerol is present at about 20 wt % of the topical anhydrous composition, and hydroxypropyl cellulose is present at about 0.75 wt % of the topical anhydrous composition.

In some embodiments, the topical anhydrous composition comprises erlotinib at about 4 wt % of the topical anhydrous composition, isopropyl alcohol is present at about 15 wt % of the topical anhydrous composition, PEG400 is present at about 55.25 wt % of the topical anhydrous composition, diisopropyl adipate is present at about 15 wt % of the topical anhydrous composition, glycerol is present at about 10 wt % of the topical anhydrous composition, and hydroxypropyl cellulose is present at about 0.75 wt % of the topical anhydrous composition.

In some embodiments, the topical anhydrous composition comprises erlotinib at about 3.9 wt % of the topical anhydrous composition, isopropyl alcohol is present from about 10 wt % to about 20 wt % of the topical anhydrous composition, PEG400 is present from about 50 wt % to about 60 wt % of the topical anhydrous composition, diisopropyl adipate is present from about 10 wt % to about 20 wt % of the topical anhydrous composition, glycerol is present from about 5 wt % to about 15 wt % of the topical anhydrous composition, hydroxypropyl cellulose is present from about 0.1 wt % to about 5.75 wt % of the topical anhydrous composition, propyl gallate is present from about 0.001 wt % to about 5 wt % of the topical anhydrous composition, ascorbyl palmitate is present from about 0.001 wt % to about 5 wt % of the topical anhydrous composition, and a-tocopherol is present from about 0.001 wt % to about 5 wt % of the topical anhydrous composition.

In some embodiments, the topical anhydrous composition comprises erlotinib at about 3.9 wt % of the topical anhydrous composition, isopropyl alcohol is present from about 10 wt % to about 20 wt % of the topical anhydrous composition, PEG400 is present from about 46 wt % to about 56 wt % of the topical anhydrous composition, propylene glycol is present from about 0.1 wt % to about 6.5 wt % of the topical anhydrous composition, diisopropyl adipate is present from about 10 wt % to about 20 wt % of the topical anhydrous composition, glycerol is present from about 5 wt % to about 15 wt % of the topical anhydrous composition, benzyl alcohol is present from about 0.1 wt % to about 7 wt % of the topical anhydrous composition, oleyl alcohol is present from about 0.1 wt % to about 5.75 wt % of the topical anhydrous composition, hydroxypropyl cellulose is present from about 0.1 wt % to about 5.75 wt % of the topical anhydrous composition, propyl gallate is present from about 0.001 wt % to about 5 wt % of the topical anhydrous composition, ascorbyl palmitate is present from about 0.001 wt % to about 5 wt % of the topical anhydrous composition, and a-tocopherol is present from about 0.001 wt % to about 5 wt % of the topical anhydrous composition.

In some embodiments, the topical anhydrous composition comprises erlotinib at about 3.9 wt % of the topical anhydrous composition, PEG400 is present from about 23.3 wt % to about 33.3 wt % of the topical anhydrous composition, propylene glycol is present from about 10 wt % to about 20 wt % of the topical anhydrous composition, Transcutol P is present from about 10 wt % to about 20 wt % of the topical anhydrous composition, diisopropyl adipate is present from about 10 wt % to about 20 wt % of the topical anhydrous composition, glycerol is present from about 5 wt % to about 15 wt % of the topical anhydrous composition, benzyl alcohol is present from about 0.1 wt % to about 7 wt % of the topical anhydrous composition, oleyl alcohol is present from about 5 wt % to about 15 wt\hat{ } % of the topical anhydrous composition, hydroxypropyl cellulose is present from about 0.1 wt % to about 5.75 wt % of the topical anhydrous composition, propyl gallate is present from about 0.001 wt % to about 5 wt % of the topical anhydrous composition, ascorbyl palmitate is present from about 0.001 wt % to about 5 wt % of the topical anhydrous composition, and a-tocopherol is present from about 0.001 wt % to about 5 wt % of the topical anhydrous composition.

In some embodiments, the topical anhydrous composition comprises erlotinib at about 3.26 wt % of the topical anhydrous composition, PEG400 is present at about 55.668 wt % of the topical anhydrous composition, isopropyl alcohol is present at about 15 wt % of the topical anhydrous composition, propylene glycol is present at about 15 wt % of the topical anhydrous composition, glycerol is present at about 10 wt % of the topical anhydrous composition, hydroxypropyl cellulose is present at about 1 wt % of the topical anhydrous composition, propyl gallate is present at about 0.05 wt % of the topical anhydrous composition, ascorbyl palmitate is present at about 0.02 wt % of the topical anhydrous composition, and a-tocopherol is present at about 0.002 wt % of the topical anhydrous composition.

In some embodiments, the topical anhydrous composition comprises erlotinib at about 3.26 wt % of the topical anhydrous composition, PEG400 is present at about 55.388 wt % of the topical anhydrous composition, isopropyl alcohol is present at about 15 wt % of the topical anhydrous composition, propylene glycol is present at about 15 wt % of the topical anhydrous composition, glycerol is present at about 10 wt % of the topical anhydrous composition, hydroxypropyl cellulose is present at about 1 wt % of the topical anhydrous composition, propyl gallate is present at about 0.05 wt % of the topical anhydrous composition, ascorbyl palmitate is present at about 0.02 wt % of the topical anhydrous composition, and a-tocopherol is present at about 0.002 wt % of the topical anhydrous composition.

In some embodiments, the topical anhydrous composition comprises erlotinib at about 4.54 wt % of the topical anhydrous composition, PEG400 is present at about 44.388 wt % of the topical anhydrous composition, isopropyl alcohol is present at about 15 wt % of the topical anhydrous composition, propylene glycol is present at about 25 wt % of the topical anhydrous composition, glycerol is present at about 10 wt % of the topical anhydrous composition, hydroxypropyl cellulose is present at about 1 wt % of the topical anhydrous composition, propyl gallate is present at about 0.05 wt % of the topical anhydrous composition, ascorbyl palmitate is present at about 0.02 wt % of the topical anhydrous composition, and a-tocopherol is present at about 0.002 wt % of the topical anhydrous composition.

In some embodiments, the topical anhydrous composition comprises erlotinib at about 4.0 wt % of the topical anhydrous composition, PEG400 is present at about 55.4 wt % of the topical anhydrous composition, isopropyl alcohol is present at about 15 wt % of the topical anhydrous composition, diisopropyl adipate is present at about 15 wt % of the topical anhydrous composition, glycerol is present at about 10 wt % of the topical anhydrous composition, hydroxypropyl cellulose is present at about 0.75 wt % of the topical anhydrous composition, propyl gallate is present at about 0.05 wt % of the topical anhydrous composition, ascorbyl palmitate is present at about 0.02 wt % of the topical anhydrous composition, and a-tocopherol is present at about 0.002 wt % of the topical anhydrous composition.

In some embodiments, the topical anhydrous composition comprises erlotinib at about 4.51 wt % of the topical anhydrous composition, PEG400 is present at about 54.418 wt % of the topical anhydrous composition, ethanol is present at about 15 wt % of the topical anhydrous composition, diisopropyl adipate is present at about 15 wt % of the topical anhydrous composition, glycerol is present at about 10 wt % of the topical anhydrous composition, hydroxypropyl cellulose is present at about 1 wt % of the topical anhydrous composition, propyl gallate is present at about 0.05 wt % of the topical anhydrous composition, ascorbyl palmitate is present at about 0.02 wt % of the topical anhydrous composition, and a-tocopherol is present at about 0.002 wt % of the topical anhydrous composition.

In some embodiments, the topical anhydrous composition comprises erlotinib at about 2.384 wt % of the topical anhydrous composition, PEG400 is present at about 56.548 wt % of the topical anhydrous composition, isopropyl alcohol is present at about 15 wt % of the topical anhydrous composition, propylene glycol is present at about 15 wt % of the topical anhydrous composition, glycerol is present at about 10 wt % of the topical anhydrous composition, hydroxypropyl cellulose is present at about 1 wt % of the topical anhydrous composition, propyl gallate is present at about 0.05 wt % of the topical anhydrous composition, ascorbyl palmitate is present at about 0.02 wt % of the topical anhydrous composition, and a-tocopherol is present at about 0.002 wt % of the topical anhydrous composition.

In some embodiments, the topical anhydrous composition comprises erlotinib at about 2.69 wt % of the topical anhydrous composition, PEG400 is present at about 26.238 wt % of the topical anhydrous composition, isopropyl alcohol is present at about 15 wt % of the topical anhydrous composition, propylene glycol is present at about 15 wt % of the topical anhydrous composition, diisopropyl adipate is present at about 15 wt % of the topical anhydrous composition, hydroxypropyl cellulose is present at about 1 wt % of the topical anhydrous composition, propyl gallate is present at about 0.05 wt % of the topical anhydrous composition, ascorbyl palmitate is present at about 0.02 wt % of the topical anhydrous composition, and a-tocopherol is present at about 0.002 wt % of the topical anhydrous composition.

In some embodiments, the topical anhydrous composition comprises erlotinib at about 3.254 wt % of the topical anhydrous composition, PEG400 is present at about 47.608 wt % of the topical anhydrous composition, isopropyl alcohol is present at about 15 wt % of the topical anhydrous composition, propylene glycol is present at about 15 wt % of the topical anhydrous composition, glycerol is present at about 10 wt % of the topical anhydrous composition, hydroxypropyl cellulose is present at about 1 wt % of the topical anhydrous composition, propyl gallate is present at about 0.05 wt % of the topical anhydrous composition, ascorbyl palmitate is present at about 0.02 wt % of the topical anhydrous composition, and a-tocopherol is present at about 0.002 wt % of the topical anhydrous composition.

In some embodiments, the topical anhydrous composition comprises erlotinib at about 1.0 wt % of the topical anhydrous composition, PEG400 is present at about 62.9 wt % of the topical anhydrous composition, isopropyl alcohol is present at about 10 wt % of the topical anhydrous composition, glycerol is present at about 10 wt % of the topical anhydrous composition, diisopropyl adipate is present at about 15 wt % of the topical anhydrous composition, hydroxypropyl cellulose is present at about 1 wt % of the topical anhydrous composition, and butylated hydroxytoluene is present at about 0.1 wt % of the topical anhydrous composition.

In some embodiments, the topical anhydrous composition comprises erlotinib at about 3.0 wt % of the topical anhydrous composition, PEG400 is present at about 60.9 wt % of the topical anhydrous composition, isopropyl alcohol is present at about 10 wt % of the topical anhydrous composition, glycerol is present at about 10 wt % of the topical anhydrous composition, diisopropyl adipate is present at about 15 wt % of the topical anhydrous composition, hydroxypropyl cellulose is present at about 1 wt % of the topical anhydrous composition, and butylated hydroxytoluene is present at about 0.1 wt % of the topical anhydrous composition.

In some embodiments, the topical anhydrous composition comprises erlotinib at about 4.0 wt % of the topical anhydrous composition, PEG400 is present at about 59.9 wt % of the topical anhydrous composition, isopropyl alcohol is present at about 10 wt % of the topical anhydrous composition, glycerol is present at about 10 wt % of the topical anhydrous composition, diisopropyl adipate is present at about 15 wt % of the topical anhydrous composition, hydroxypropyl cellulose is present at about 1 wt % of the topical anhydrous composition, and butylated hydroxytoluene is present at about 0.1 wt % of the topical anhydrous composition.

In some embodiments, the topical anhydrous compositions comprises an effective amount of one or more EGFR inhibitors, one or more solvents, one or more antioxidants, and no gelling agents.

In some embodiments, the topical anhydrous compositions of EGFR inhibitor further comprises a polymeric surfactant, a moisturizing agent, a cooling agent, a rheology modifier, a pH adjusting agent, a preservative, and combinations thereof.

In some embodiments, the topical anhydrous compositions of EGFR inhibitor comprises one or more polymeric surfactants. Polymers having surfactant properties (polymeric surfactant) can be, but are not limited to, hydrophobically modified polyacrylic acid (trade name Pemulen™ TR-1 and TR-2), copolymers based on acrylamidoalkyl sulfonic acid and cyclic N-vinylcarboxamides (tradename Aristoflex® AVC), copolymers based on acrylamidoalkyl sulfonic acid and hydrophobically modified methacrylic acid (tradename Aristoflex® HMB), and a homopolymer of acrylamidoalkyl sulfonic acid (tradename Granthix APP). Another class of notable polymeric emulsifier includes hydrophobically-modified, crosslinked, anionic acrylic copolymers, including random polymers, but may also exist in other forms such as block, star, graft, and the like. In one embodiment, the hydrophobically modified, crosslinked, anionic acrylic copolymer may be synthesized from at least one acidic monomer and at least one hydrophobic ethylenically unsaturated monomer. Examples of suitable acidic monomers include those ethylenically unsaturated acid monomers that may be neutralized by a base. Examples of suitable hydrophobic ethylenically unsaturated monomers include those that contain a hydrophobic chain having a carbon chain length of at least about 3 carbon atoms. Other materials that may be suitable polymeric surfactants can include ethylene oxide/propylene oxide block copolymers, sold under the trade name PLURONIC®, modified cellulose polymers such as those modified cellulose polymers described by the trade name KLUCEL® (hydroxypropyl cellulose), monomeric anionic surfactants, monomeric amphoteric surfactants, betaine, and combinations thereof. Other suitable polymeric surfactants include copolymers based on acrylamidoalkylsulfonic acids and cyclic N-vinylcarboxamides and/or linear N-vinylcarboxamides (e.g., Aristoflex® AVC and Aristoflex® HMB) and a betaine. In preferred embodiments, the polymeric surfactants include poloxamer P-188, poloxamer P-138, poloxamer P-237, poloxamer P-288, poloxamer P-124, poloxamer P-338, poloxamer P-407, D-a-Tocopheryl polyethylene glycol 1000 succinate, Brij 020, and combinations thereof. In some embodiments, the polymeric surfactant is present from about 0.1 wt % to about 50 wt % of the topical anhydrous composition, about 0.1 wt % to about 40 wt % of the topical anhydrous composition, about 0.1 wt % to about 30 wt % of the topical anhydrous composition, about 0.1 wt % to about 20 wt % of the topical anhydrous composition, or about 0.1 wt % to about 10 wt % of the topical anhydrous composition.

In some embodiments, the topical anhydrous compositions of EGFR inhibitor may further comprise one or more moisturizing agents or an emollient component, for example mineral oil, dimethicone, cyclomethicone, cholesterol, or combinations thereof. In some embodiments, the topical anhydrous composition includes liquid emollients such as polyhydric alcohols, polyols, saccharides, triglycerides, hydrocarbons, silicones, fatty acids, fatty, esters, fatty alcohols, and blends thereof. In some embodiments, the moisturizing agent is present from about 0.5 wt % to about 10 wt % of the topical anhydrous composition, about 0.5 wt % to about 8 wt % of the topical anhydrous composition, about 0.5 wt % to about 6 wt % of the topical anhydrous composition, about 0.5 wt % to about 4 wt % of the topical anhydrous composition, or about 0.5 wt % to about 1 wt % of the topical anhydrous composition.

In some embodiments, the topical anhydrous compositions disclosed herein does not contain water. In some embodiments, the topical anhydrous compositions disclosed herein contains substantially no water. In some embodiments, the topical anhydrous compositions disclosed herein contain less than 1% of water in the topical anhydrous composition. In some embodiments, the topical anhydrous compositions disclosed herein contain less than 0.5% of water in the topical anhydrous composition. In some embodiments, the topical anhydrous compositions disclosed herein contain less than 0.1% of water in the topical anhydrous composition.

In some embodiments, the topical anhydrous compositions of EGFR inhibitors further comprise a rheology modifier, a pH adjusting agent, a preservative, and combinations thereof.

The compositions of the present invention also can further comprise a polymer having thickening properties (rheology modifier). In one embodiment, the polymer having thickening properties can be a hydrophobically modified cross-linked acrylate copolymer (Carbopol® Ultrez 20). Other polymers having similar properties may also be used. Non-limiting examples of polymers having thickening properties can include PEG-150 distearate, PEG-7 glyceryl cocoate, PEG-200 hydrogenated glyceryl palmitate, PEG-120 methyl glucose dioleate, carboxymethylene polymer, carboxyvinyl polymer, acrylates, C10-C30 alkyl acrylate crosspolymers, isopropyl myristate, and combinations thereof. In some embodiments, the polymer having thickening properties can comprise about 0.1 wt % to about 3 wt %. In another embodiment, polymers having thickening properties can be present in amounts of 0.4 wt % to about 1.0 wt % of the topical anhydrous composition. In one embodiment, the polymer having thickening properties comprises about 0.5 wt % to about 0.75 wt % of the topical anhydrous composition. The thickening polymer can be mixed with the surfactant polymer in some embodiments.

In some embodiments, the compositions of the present invention can further comprise a non-aqueous pH adjusting agent or a non-aqueous buffering agent, which is present in the composition to neutralize and/or activate the thickening polymer in order to facilitate the formation of a composition having the desirable rheological qualities. Any anhydrous base or buffer system known in the art and suitable for use in a skin contact application can be used. In one embodiment, the base can include triethanolamine, tetrasodium ethylenediaminetetraacetic acid (EDTA), alkali metal hydroxides like sodium hydroxide (NaOH), salts of weak acids such as ammonium lactate, sodium citrate, sodium ascorbate, or mixtures thereof. The base component also provides utility in that the apparent pH of the overall composition may be adjusted to a range favorable for minimizing irritation of the skin due to pH effects. In some embodiments, compositions of the present invention can also include anhydrous acids or the acid component of a buffer system, and any acid known in the art and appropriate for human skin contact may be used. Examples of acids useful in the present composition and commonly used to adjust pH of topical compositions include but are not limited to: citric acid, lactic acid, ascorbic acid, tartaric acid, and hydrochloric acid, and combinations of these and similar acids. Specific examples of the pH levels of the composition include about pH 4, about pH 4.5, about pH 5, about pH 5.6, about pH 6, about pH 7, about pH 7.4, about pH 8, and ranges between any two of these values.

Compositions disclosed herein may further comprise preservatives to prevent the growth of harmful microorganisms. While it is in the aqueous phase that microorganisms tend to grow, microorganisms can also reside in the oil phase. As such, preservatives which have solubility in oil are preferably employed in the present compositions. Generally, from one tenth of one percent by weight to one percent by weight of preservatives are adequate. The traditional preservatives for cosmetics and pharmaceuticals are alkyl esters of para-hydroxybenzoic acid. Other preservatives which have more recently come into use include hydantoin derivatives, propionate salts, cationic surfactants such as benzalkonium chloride; benzyl alcohol, sorbic acid, and a variety of quaternary ammonium compounds. Cosmetic chemists are familiar with appropriate preservatives and routinely choose them to satisfy the preservative challenge test and to provide product stability. Particularly preferred preservatives for a preferred anhydrous composition of this invention are phenoxyethanol, phenethyl alcohol, methyl and propyl parahydroxybenzoates, imidazolidinyl urea, and quaternium-15. The preservatives should be selected having regard for the use of the composition and possible incompatibilities between the preservatives and the other ingredients in the composition.

In some embodiments, the topical anhydrous compositions are sustained release compositions for controlled release of EGFR inhibitors in order to diminish rapid uptake and systemic absorption of the applied agent. Sustained (or controlled) release refers to the gradual release of EGFR inhibitors from the composition over a period of time. While there may be an initial burst phase, in some embodiments, it is preferred that the release display relatively linear kinetics, thereby providing a constant supply of the EGFR inhibitor over the release period. The release period may vary from about 1 hour to about 8 hours, depending upon the skin disorder and its intended use. The compositions may further comprise various biodegradable polymers to facilitate slow release, such as poly-lactides (PLA), poly-glycolides (PGA), poly butylene succinate (PBS), polyhydroxyalkanoate (PHA), polycaprolactone acid lactone (PCL), polyhydroxybutyrate (PHB), glycolic amyl (PHV), PHB and PHV copolymer (PHBV), and poly lactic acid (PLA)-polyethylene glycol (PEG) copolymers (PLEG). In some embodiments, the preferred polymer is Pluronic® 127.

In some embodiments, the topical anhydrous composition is a nanoparticle formulation comprising an EGFR inhibitor and a carrier protein. In some embodiments, the topical anhydrous composition is a nanoparticle formulation comprising erlotinib and albumin. In some embodiments, the nanoparticle formulation does not contain a carrier protein. In some embodiments, the average particle size of the nanoparticle is from about 10 nm to about 150 nm. In some embodiments, the topical anhydrous composition comprises an EGFR inhibitor bound to albumin nanoparticle, such as ABI-009.

In some embodiments, the viscosity of the topical anhydrous compositions disclosed herein is generally that of a thick liquid or gel but can reach a paste like consistency. Generally, the viscosity is a minimum of about 5,000, 10,000 or 15,000 preferably about 20,000 to a maximum of about 12,000,000, 2,000,000 or even about 600,000 cP.

The topical anhydrous composition of EGFR inhibitors may comprise further ingredients as required. For example, it may contain a further active ingredient, e.g. a corticosteroid, an antibiotic, an antimycotic, an antiviral agent, a mTOR inhibitor, a hedgehog pathway inhibitor, and combinations thereof. Moreover, it may comprise one or more further excipients, such as permeation enhancers DMSO, Transcutol®, menthol, oleic acid, n-alkanols, 1-alkyl-2-pyrrolidones, N,N-dimethlyalkanamides, and 1,2-alkanediols, and the like.

In some embodiments, the mTOR inhibitors are selected from rapamycin (sirolimus), everolimus, ridaforolimus, temsirolimus, zotarolimus, rapamycin prodrug AP-23573, AP-23481, torin-1, torin-2, WYE-354, dactolisib, voxtalisib, omipalisib, apitolisib, vistusertib, gedatolisib, WYE-125132, BGT226, palomid 529, GDC-0349, XL388, CZ415, CC-223, SF1126, INK128, AZD8055, biolimus A9 (umirolimus), GSK2126458, OSI027, PP121, WYE-687, WAY-600, PI-103, KU-0063794, Torkinib (PP242), PF-04691502, RTB101, TAM-01, TAM-03, LY294002, and pharmaceutically acceptable salts, hydrates, solvates, polymorphs, or amorphous solids thereof.

In some embodiments, the sonic hedgehog pathway inhibitors include inhibitors of Shh, PTCH, SMO, GLI1-3, SOX9, and downstream targets. Non-limiting examples of hedgehog pathway inhibitors include GDC-0449 (vismodegib/Erivedge®), Odomzo® (sonidegib, LDE225, erismodegib), BMS-833923/XL139, PF-04449913 (glasdegib), LY2940680 (taladegib), IPI-926 (saridegib), arsenic trioxide (ATO), cyclopamine, CUR61414, PF-5274857, TAK-441, MRT-92, Jervine, GANTs, RU-SK/43-129/130, Shh Monoclonal Antibody 5E1-135, and a triazole antifungal agent, such as itraconazole, and combinations thereof.

In some embodiments, the compositions may further comprise other skin care agents, including, but not limited to, angiotensin converting enzyme inhibitors such asbenazepril, lotensin, captopril, enalapril, fosinopril, lisinopril, moexipril perindopril quinapril, retinol, steroids, sunblock, salicylate, minocycline, antifungals, peptides, antibodies, lidocaine, and the like and combinations thereof. In some embodiments, other skin care agents include N-acyl amino acid compounds including, for example, N-acyl phenylalanine, N-acyl tyrosine, and the like, their isomers, including their D and L isomers, salts, derivatives, and mixtures thereof. An example of a suitable N-acyl amino acid is N-undecylenoyl-L-phenylalanine is commercially available under the tradename SEPIWHITE®. Other skin active agents include, but are not limited to, Lavandox, Thallasine 2, Argireline NP, Gatuline In-Tense and Gatuline Expression, Myoxinol LS 9736, Syn-ake, and Instensyl®, Sesaflash™, N-acetyl D-glucosamine, panthenol (for example, DL panthenol available from Alps Pharmaceutical Inc.), tocopheryl nicotinate, benzoyl peroxide, 3-hydroxy benzoic acid, flavonoids (for example, flavanone, chalcone), farnesol, phytantriol, glycolic acid, lactic acid, 4-hydroxy benzoic acid, acetyl salicylic acid, 2-hydroxybutanoic acid, 2-hydroxypentanoic acid, 2-hydroxyhexanoic acid, 13-cis-retinoic acid, 9-cis-retinoic acid, all-trans retinoic acid, adapalene, tazarotene, acitretin, retinol, retinyl esters (for example, retinyl propionate), phytic acid, N-acetyl-L-cysteine, lipoic acid, tocopherol and its esters (for example, tocopheryl acetate: DL-a-tocopheryl acetate available from Eisai), azelaic acid, arachidonic acid, tetracycline, ibuprofen, naproxen, ketoprofen, hydrocortisone, acetominophen, resorcinol, phenoxyethanol, phenoxypropanol, phenoxyisopropanol, 2,4,4′-trichloro-2′-hydroxy diphenyl ether, 3,4,4′-trichlorocarbanilide, octopirox, lidocaine hydrochloride, clotrimazole, miconazole, ketoconazole, neomycin sulfate, theophylline, and mixtures thereof.

One or more sunscreens may be incorporated into the present anhydrous compositions. A variety of sunscreens may be employed including the p-aminobenzoic acid derivatives such as p-(2-ethylhexyl)dimethylaminobenzoate, and benzophenone derivatives such as (2-hydroxy-4-methoxyphenyl)phenylmethanone, Mexoryl™ SX, and Mexoryl™ XL, terephthalylidene dicamphor sulfonic acid, and drometrizole trisiloxane. Other non-limiting examples include benzophenones (oxybenzone and sulisobenzone), cinnamates (octylmethoxy cinnamate and cinoxate), salicylates (homomethyl salicylate) anthranilates, TiO2, avobenzone, bemotrizinol, bisoctrizole, 3-(4-methylbenzylidene)-camphor, cinoxate, diethylamino hydroxybenzoyl hexyl benzoate, dioxybenzone, drometrizole trisiloxane, ecamsule, ethylhexyl triazone, homosalate, menthyl anthranilate, octocrylene, octyl salicylate, iscotrizinol, isopentenyl-4-methoxycinnamate, octyl-dimethyl-p-aminobenzoic acid, octyl-methoxycinnamate, oxybenzone, polysilicone-15, trolamine salicylate, and ZnO. The exact amount of sunscreen employed in the present compositions will vary depending on the degree of protection desired from the sun's harmful rays.

The topical anhydrous compositions of the invention may also comprise one or more pigments to color the composition, and a fragrance, such as Firmenich and Co. 66.001/NY/G fragrance oil, to make the composition soothing to the olfactory system. The amount of these ingredients present in the composition will depend on the specific effect desired.

In embodiments, the topical anhydrous compositions may be in solid dosage forms including, but not limited to, topical dosage forms including, but not limited to, solutions, powders, fluid suspensions, semi-solids, ointments, pastes, creams, lotions, gels, jellies, and foams; and parenteral dosage forms including, but not limited to, solutions, suspensions, and dry powders. The active ingredients can be contained in such compositions with pharmaceutically acceptable diluents, fillers, disintegrants, binders, lubricants, surfactants, hydrophobic vehicles, emulsifiers, buffers, humectants, moisturizers, solubilizers, preservatives and the like. Pharmaceutical compositions of the compounds also can include suitable solid or gel phase carriers or excipients. Examples of such carriers or excipients include but are not limited to calcium carbonate, calcium phosphate, gelatin, and polymers such as, for example, polyethylene glycols.

In some embodiments, the topical anhydrous compositions disclosed herein may be in the form of a paste, a liquid, lotion, spray, aerosol, powder, ointment, cream, mouthwash, toothpaste, foam, gel, a solid stick, and combinations thereof. In some embodiments, the compositions disclosed herein are easy to spread, quick absorption, moisturizing, non-greasy, non-irritating to patients' skin, aesthetically pleasing to use, and has cooling effect.

In embodiments, the compositions described herein may be formulated as a liquid. Liquid dosage forms for topical administration may include diluents such as, for example, alcohols, glycols, oils, and the like. Traditional ointment bases (i.e. carrier) include hydrocarbons (petrolatum, beeswax, etc.) vegetable oils, fatty alcohols (cholesterol, lanolin, wool alcohol, stearyl alcohol, etc.) or silicones. Insoluble solids such as starch, zinc oxide, calcium carbonate, or talc can also be used in ointments and creams. Gel forms of the compositions may contain a network of polymers or colloidal solid particles. Such polymers or colloids (gelling or thickening agents) are typically present at concentrations of less than 10% w/w and include carboxymethyl cellulose, hydroxypropylmethyl cellulose, hydroxyethyl cellulose, methyl cellulose, sodium alginate, alginic acid, pectin, tragacanth, carrageen, agar, clays, aluminum silicate, carbomers, and the like.

In some embodiments, the EGFR inhibitors in the compositions disclosed herein are stable for extended periods of time. For example, in some embodiments, the EGFR inhibitors in the compositions are stable at temperature ranges from about 4° C. to about 50° C. for a period of 12-36 months. In some embodiments, the EGFR inhibitors in the compositions are stable at temperature ranges from about 4° C. to about 45° C. for a period of 12-36 months. In some embodiments, the EGFR inhibitors in the compositions are stable at temperature ranges from about 4° C. to about 40° C. for a period of 12-36 months. In some embodiments, the EGFR inhibitors in the compositions are stable at temperature ranges from about 4° C. to about 35° C. for a period of 12-36 months. in some embodiments, the EGFR inhibitors in the compositions are stable at temperature ranges from about 4° C. to about 30° C. for a period of 12-36 months.

Also disclosed herein are methods to treat a skin disorder in a subject. In some embodiments, a method of treating a skin disorder in a subject comprises topically administering an effective amount of a topical anhydrous composition comprising an effective amount of one or more EGFR inhibitors, one or more solvents, one or more gelling agents, and one or more antioxidants.

In some embodiments, a method of treating a skin disorder in a subject comprises topically administering an effective amount of a topical anhydrous composition comprising an effective amount of one or more EGFR inhibitors, one or more solvents, and one or more antioxidants.

Non-limiting examples of skin disorder that may be treated by the topical anhydrous compositions include plantar hyperkeratosis, blisters, tuberous sclerosis, seborrheic keratosis, keratosis pilaris, epidermolysis bullosa, multiple minute digitate hyperkeratosis, hyperkeratosis lenticularis perstans, stasis dermatitis, focal acral hyperkeratosis, follicular hyperkeratosis, lichenoid keratoses (lichen planus, lichen sclerosus), chronic erosive oral lichen, Conradi-Eltinermann, epidermolytic ichthyosis, erythrokeratoderma variabilis, ichthyosis hystrix, KID syndrome, Netherton syndrome, Olmsted syndrome, Refsum disease, Sjogren-Larsson Syndrome, actinic keratosis, pachyonychia congenita, hyperhidrosis, warts, calluses, dermatitis (contact dermatitis, drug-induced dermatitis, allergic dermatitis, nummular dermatitis, perioral dermatitis, neurodermatitis, seborrheic dermatitis, and atopic dermatitis), psoriasis, acne, carbunculosis, cellulitis, furunculosis, granuloma, acanthosis nigricans, athlete's foot, bacterial vaginosis, balanitis, dermatofibrosarcoma protruberans, basal cell carcinoma, squamous cell carcinoma, melanoma, merkel cell carcinoma, keloid, cystic lymphangioma, Cavernous lymphangioma, venous malformation, epidermal nevi, bromhidrosis, dermatophytosis, candidiasis, onychomycosis, tinea (tinea alba, tinea pedis, tinea unguium, tinea manuum, tinea cruris, tinea corporis, tinea capitis, tinea faciei, tinea barbae, tinea imbricata, tinea nigra, tinea versicolor, tinea incognito), eczema, dyshydrotic eczema, decubitous ulcer, ecthyma, erysipalus, erythema multiforme, impetigo, insect bites, genital warts, hemangioma, herpes, hives, hyperhidrosis, filariasis, lentigines, lupus, miliaria, milker's nodules, molluscum contagiosum, myiasis, scabies, cutaneous larva migrans, furuncular myiasis, migratory myiasis, pediculosis, nevus araneus, panniculitis, paronychia, pemphigoid, pityriasis, pruritis vulvae, rosacea, trichomoniasis, vaginal yeast infection, vitiligo, xeroderma, angiofibroma, Bannayan-Riley-Ruvalcaba syndrome, basal cell nevus syndrome (Gorlin syndrome), Birt-Hogg-Dube syndrome, Blue rubber bleb nevus syndrome, Cowden disease, cutaneous T-cell lymphoma, diffuse microcystic lymphatic malformations, epidermolysis bullosa simplex, extramammary paget, familial multiple discoid fibromas, Hailey-Hailey disease, infantile hemangiomas, juvenile polyposis syndrome, Kaposi sarcoma, Kaposiform hemangioendothelioma, Keloid scar disease, Lhermitte-Duclos syndrome, metastatic melanoma, Muir-Tone syndrome, neurofibromatosis, nonmelanoma skin cancer, oral graft-versus-host disease, Pemphigus vulgaris, Peutz-Jeghers syndrome, Port-wine stains, Proteus syndrome, Proteus-like Syndrome, refractory hemangioendotheliomas in Maffucci syndrome, Sturge-weber syndrome, hereditary footpad hyperkeratosis (HFH) in canines, cutaneous sarcoidosis, cutaneous Castleman Disease, Bullous Pemphigoid, pyogenic granulomas, essential telangiectasias, cherry angiomas, Brooke-Speigler syndrome, nevus sebaceous, epidermal nevus, chelitis granulomatosis, overgrowth syndromes, gingival hypertrophy, xeroderma pigmentosum, cutaneous acute graft-versus-host disease, cutaneous chronic graft-versus-host disease, sclerodermatous graft-versus-host disease, epidermolytic palmoplantar keratoderma, cutaneous neurofibromatosis, multiple trichoepitheliomas, Darier's disease (also known as keratosis follicularis or Darier-White disease), epidermolysis, ichthyosis, lamellar ichthyosis, Bowen disease and combinations thereof.

In some embodiments, the skin disorder that is treated is angiofibroma. In some embodiments, the skin disorder that is treated is pachyonychia congenita. In some embodiments, a symptom of pachyonychia congenita is treated and the symptom is selected from pain, itch or a combination thereof.

In some embodiments, the skin disorder that is treated is Hailey-Hailey disease. In some embodiments, the skin disorder that is treated is Darier's disease (also known as Keratosis follicularis or Darier-White disease). In some embodiments, the skin disorder that is treated is epidermolysis. In some embodiments, the skin disorder that is treated is epidermolysis bullosa simplex. In some embodiments, the skin disorder that is treated is Olmsted Syndrome. In some embodiments, the skin disorder that is treated is ichthyosis. In some embodiments, the skin disorder that is treated is epidermolytic ichthyosis. In some embodiments, the skin disorder that is treated is lamellar ichthyosis. In some embodiments, the skin disorder that is treated is acanthosis nigricans.

In some embodiments, a method of treating Hailey-Hailey disease in a subject comprises topically administering an effective amount of a topical anhydrous composition comprising an effective amount of one or more EGFR inhibitors, one or more solvents, one or more gelling agents, and one or more antioxidants.

In some embodiments, a method of treating Darier's disease (also known as keratosis follicularis or Darier-White disease) in a subject comprises topically administering an effective amount of a topical anhydrous composition comprising an effective amount of one or more EGFR inhibitors, one or more solvents, one or more gelling agents, and one or more antioxidants

In some embodiments, a method of treating epidermolysis in a subject comprises topically administering an effective amount of a topical anhydrous composition comprising an effective amount of one or more EGFR inhibitors, one or more solvents, one or more gelling agents, and one or more antioxidants.

In some embodiments, a method of treating epidermolysis bullosa simplex in a subject comprises topically administering an effective amount of a topical anhydrous composition comprising an effective amount of one or more EGFR inhibitors, one or more solvents, one or more gelling agents, and one or more antioxidants.

In some embodiments, a method of treating Olmsted Syndrome in a subject comprises topically administering an effective amount of a topical anhydrous composition comprising an effective amount of one or more EGFR inhibitors, one or more solvents, one or more gelling agents, and one or more antioxidants.

In some embodiments, a method of treating ichthyosis in a subject comprises topically administering an effective amount of a topical anhydrous composition comprising an effective amount of one or more EGFR inhibitors, one or more solvents, one or more gelling agents, and one or more antioxidants.

In some embodiments, a method of treating epidermolytic ichthyosis in a subject comprises topically administering an effective amount of a topical anhydrous composition comprising an effective amount of one or more EGFR inhibitors, one or more solvents, one or more gelling agents, and one or more antioxidants.

In some embodiments, a method of treating lamellar ichthyosis in a subject comprises topically administering an effective amount of a topical anhydrous composition comprising an effective amount of one or more EGFR inhibitors, one or more solvents, one or more gelling agents, and one or more antioxidants.

In some embodiments, a method of treating acanthosis nigricans in a subject comprises topically administering an effective amount of a topical anhydrous composition comprising an effective amount of one or more EGFR inhibitors, one or more solvents, one or more gelling agents, and one or more antioxidants.

In some embodiments, administration of the composition is by topical application.

In some embodiments, the topical anhydrous composition of EGFR inhibitors are administered topically, and the EGFR inhibitor reaches epidermal and dermal layer through absorption and gransport. In some embodiments, the topical application of the topical anhydrous composition does not result in significant systemic absorption of the EGFR inhibitors.

In some embodiments, the topical administration of the topical anhydrous compositions results in delivery of the EGFR inhibitors to epidermis of the skin. In some embodiments, the topical administration of the topical anhydrous compositions results in delivery of EGFR inhibitors to epidermis and dermis.

In some embodiments, the method of treating a skin disorder involves administering topically a topical anhydrous composition that includes one or more EGFR inhibitors present from about 0.1 wt % to about 20 wt % of the topical anhydrous composition, one or more solvents present from about 1 wt % to about 99 wt % of the topical anhydrous composition, one or more gelling agents present from about 0.1 wt % to about 5 wt % of the topical anhydrous composition, and one or more antioxidants present from about 0.001 wt % to about 1 wt % of the topical anhydrous composition. In some embodiments, the composition may further include a polymeric surfactant, a moisturizing agent, a rheology modifier, a pH adjusting agent, a preservative, and combinations thereof. In some embodiments, the topical anhydrous compositions do not contain gelling agents.

In some embodiments, the topical anhydrous compositions can be topically applied to the skin, preferably by manually rubbing the applied amount over the skin to thoroughly coat the skin. The rubbing action preferably is a gentle rubbing or massaging for a period of at least about 5 second, preferably about 5 to about 30 seconds to spread all over the skin. The moisture or water present on the skin may emulsify the topical anhydrous composition due to continuous rubbing and massaging, resulting in the formation of an emulsion in situ on the skin.

Some embodiments of the invention are directed to a method of treating hair loss in a subject. In some embodiments, the method of treating hair loss includes administering to the subject in need thereof an effective amount of a topical anhydrous composition comprising an effective amount one or more EGFR inhibitors, one or more solvents, one or more gelling agents, and one or more antioxidants. In embodiments, treatment of diseases related to hair, hair shaft, hair follicles, hair bulbs, oil glands, and components thereof, include, for example, hair loss, dandruff, seborrheic dermatitis, alopecia areata, hair disease, ringworm, tinea capitis, folliculitis, pattern hair loss, telogen effluvium, cradle cap, trichotillomania, traction alopecia, trichorrhexis nodosa, folliculitis decalvans, head lice infestation, frontal fibrosing alopecia, non-scarring hair loss, pityriasis amiantacea, dissecting cellulitis of the scalp, acne keloidalis nuchae, monilethrix, pediculosis, alopecia totalis, pseudopelade of Brocq, bubble hair deformity, hair casts, hypertrichosis, ingrown hair, monilethrix, premature greying of hair, pattern hair loss, trichorrhexis invaginata, and the like.

The compositions disclosed herein may be applied topically to a selected area of the body from which it is desired to reduce hair growth. For example, the composition can be applied to the face, particularly to the beard area of the face, i.e., the cheek, neck, upper lip, and chin. The composition also may be used as an adjunct to other methods of hair removal including shaving, waxing, mechanical epilation, chemical depilation, electrolysis and laser-assisted hair removal. Other actions that make their concept appearance are concurrent skin benefits in addition to hair reduction. The composition can also be applied to the legs, arms, torso or armpits. The composition is suitable, for example, for reducing the growth of unwanted hair in women. In humans, the composition may be applied once or twice a day, or even more frequently, to achieve a perceived reduction in hair growth. Reduction in hair growth is demonstrated when, for example, the rate of hair growth is slowed, the need for removal is reduced, the subject perceives less hair on the treated site, or quantitatively, when the weight of hair removed (i.e., hair mass) is reduced.

Some embodiments of the invention are directed to a method of treating dry eye syndrome in a subject. In some embodiments, the method of treating dry eye syndrome includes administering to the subject in need thereof an effective amount of a topical anhydrous composition comprising one or more EGFR inhibitors, one or more solvents, one or more gelling agents, and one or more antioxidants.

In some embodiments, the topical anhydrous compositions of the present invention can, for example, be applied to a plaster, patch, bandage, or a film. In some embodiments, topical delivery is aided by the use of ultrasound technology. The ultrasound energy is applied over the tissue and to assist the diffusion of the composition past the tissue.

In embodiments, the compositions disclosed herein can be in the form of transdermal patches. The transdermal patches can be in any conventional form such as, for example, a strip, a gauze, a film, and the like. Patch material may be nonwoven or woven (e.g., gauze dressing). Layers may also be laminated during processing. It may be nonocclusive or occlusive, but the latter is preferred for backing layers. The patch is preferably hermetically sealed for storage (e.g., foil packaging). The patch can be held onto the skin and components of the patch can be held together using various adhesives. For example, the transdermal patch can be in the form of a band-aid type device, or it may be packaged in a small metal or plastic “cup”, which is strapped onto the appropriate site using an adhesive, tape, or an outer fabric or leather strap, similar to that worn as part of a watch. The entire patch may be disposable or may be refillable. In some embodiments, the compositions disclosed herein can be coated on bandages, mixed with bioadhesives, or included in dressings.

In some embodiments, a hand pump may be used to dispense the EGFR inhibitor anhydrous compositions. For example, the hand pump may be configured to dispense the required dose of EGFR inhibitor within a tolerance specified by a corresponding label approved by a government regulatory agency. The hand pump may deliver 0.5-10 mL of the composition per pump action, such as 1, 2, 3, 4, or 5 mL of the composition per pump action. In some embodiments, the EGFR inhibitor compositions may be packaged along with a pharmaceutically acceptable hand pump.

In some embodiments, metered airless dose pumps may be used to dispense the topical anhydrous compositions disclosed herein. Airless type dispensing systems typically have two methods of dispensing the product, either by using a collapsible bag type design or by using a follower piston-type design. With the collapsible bag type design, a collapsing bag is attached to the dispensing pump, which progressively collapses as the contents are removed. In the piston-type design, a rigid container, usually cylindrical or oval in form, has a follower piston that progressively reduces the container volume as product is drawn out by the dispensing pump.

In other embodiments, spray dispensing systems may be used to deliver anhydrous compositions. These dispensing systems may be configured to deliver compositions with preservatives and preservative-free topical spray compositions. In some embodiments, hand pumps with continuous dispensing in 360 degrees may be employed to deliver the compositions disclosed herein. In further embodiments, a dispensing pouch with a spout for the easy and controlled squeezing of liquid and semi-solid formulations may be used. Such pouches are ideal for flexible packaging dispensing.

In some embodiments, the topical anhydrous compositions may be administered in a conventional manner by any route by which they retain activity. For example, the topical anhydrous composition of EGFR inhibitors may be administered by routes including, but not limited to, topical, transdermal, or percutaneous. Thus, modes of administration for the compounds (either alone or in combination with other pharmaceuticals) can be, but are not limited to, sublingual, or by use of vaginal creams, suppositories, pessaries, vaginal rings, rectal suppositories, and percutaneous and topical forms such as patches and creams, lotions, gels.

The particular quantity of composition administered, of course, will be determined by the particular circumstances surrounding its use, including the composition administered, the condition of the skin, the age of the user, the degree of the skin disorder, and similar considerations. For example, the dosage may depend on the particular animal treated, the age, weight, and health of the subject, the types of concurrent treatment, if any, and frequency of treatments. Many of these factors can be easily determined by one of skill in the art (e.g., by the clinician). Typically, a single application of the composition will be applied topically to cover adequately the affected area of the skin. Subsequent applications may be made as needed to deliver the desired level of EGFR inhibitors.

In some embodiments, the composition can be administered one, two, three, four, five or more times each day, and applying can be carried out for a period of at least 1 month, 2 months, 3 months, 4 months, 6 months, 8 months or 12 months.

In some embodiments, the composition may be administered once, as needed, once daily, twice daily, three times a day, once a week, twice a week, every other week, every other day, or the like for one or more dosing cycles. A dosing cycle may include administration for about 1 week, about 2 weeks, about 3 weeks, about 4 weeks, about 5 weeks, about 6 weeks, about 7 weeks, about 8 weeks, about 9 weeks, or about 10 weeks. After this cycle, a subsequent cycle may begin approximately 1, 2, 3, 4, 5, 6, 7, 8, 9, 10, 11, or 12 weeks later. The treatment regime may include 1, 2, 3, 4, 5, or 6 cycles, each cycle being spaced apart by approximately 1, 2, 3, 4, 5, 6, 7, 8, 9, 10, 11, or 12 weeks.

In embodiments, the method of treating a skin disorder comprising administering the topical anhydrous composition described herein, wherein the method does not further include any additional medical or therapeutic intervention for treatment of the skin disorder.

In embodiments, the method of treating a skin disorder comprises administering the topical anhydrous compositions described herein, wherein the EGFR inhibitor is the only active agent administered for treating the skin disorder.

In some embodiments, the methods may include a variety of additional steps including, for example, cleaning the surface tissue at the site of applying and the like.

In embodiments, the methods may further include descaling or debriding of the tissue surface before, during or after administration of the compositions described herein. In embodiments, methods for descaling or debriding tissue surface may include electromagnetic radiation, laser, dermal abrasion, chemical peel, ultrasound, heating, cooling, or by a needle.

In embodiments, the tissue surface is descaled or debrided with abrasion. Abrasion of the outer layer or epidermis of the skin (dermal abrasion) is desirable to smooth or blend scars, blemishes, or other skin conditions that may be caused by, for example, acne, sun exposure, and aging. Standard techniques used to abrade the skin have generally been separated into two fields referred to as dermabrasion and microdermabrasion. Both techniques remove portions of the epidermis called the stratum corneum, which the body interprets as a mild injury. The body then replaces the lost skin cells, resulting in a new outer layer of skin. Additionally, despite the mild edema and erythema associated with the procedures, the skin looks and feels smoother because of the new outer layer of skin.

In embodiments, the tissue surface is descaled or debrided with microdermabrasion. Microdermabrasion refers generally to a procedure in which the surface of the skin is removed due to mechanical rubbing by a handpiece emitting a stream of sand or grit. For example, a handpiece can be used to direct an air flow containing tiny crystals of aluminum oxide, sodium chloride, or sodium bicarbonate. The momentum of the grit tends to wear away two to three cell layers of the skin with each pass of the handpiece. Alternatively, new “crystal-free” microdermabrasion techniques utilize a diamond-tipped handpiece without a stream of grit.

In embodiments, the tissue surface is descaled or debrided with electromagnetic radiation, for instance using a so-called fractional laser treatment. By way of example, such methods employ electromagnetic radiation (EMR) having one or more wavelengths of between approximately 1,850 to 100,000 nanometers and with pulse widths of between approximately 1 femtosecond (1×10-15 s) to 10 milliseconds (10×10-3 s) with fluence in the range of from approximately 1 J/cm2 to 300 J/cm2. In other examples, the tissue is descaled or debrided with electromagnetic radiation having one or more wavelengths of between approximately 2,200 to 5,000 nanometers. In still other examples, the tissue is descaled or debrided with electromagnetic radiation having one or more wavelengths of between approximately 190 to 320 nanometers with fluence in the range of from 1 J/cm2 to 300 J/cm2. Optionally, conditions selected for debriding portions of the tissue minimize the coagulation zone of tissue damage, for instance by keeping the coagulation zone to a relatively small diameter surrounding the ablated void.

Electromagnetic radiation (EMR), particularly in the form of laser light or other optical radiation, has been used in a variety of cosmetic and medical applications, including uses in dermatology, dentistry, ophthalmology, gynecology, otorhinolaryngology and internal medicine. For most dermatological applications, EMR treatment can be performed with a device that delivers the EMR to the surface of the targeted tissue(s). EMR treatment is typically designed to (a) deliver one or more particular wavelengths (or a particular continuous range of wavelengths) of energy to a tissue to induce a particular chemical reaction, (b) deliver energy to a tissue to cause an increase in temperature, or (c) deliver energy to a tissue to damage or destroy cellular or extracellular structures, such as for skin remodeling. Examples of devices that have been used to treat the skin during cosmetic procedures such as skin rejuvenation include the Palomar® LuxIR, the Palomar® 1540, 1440 and 2940 Fractional Handpieces, the Reliant Fraxel® SR Laser and similar devices by Lumenis, Alma Lasers, Sciton and many others.

In embodiments, the methods may further include photodynamic therapy before, during or after administration of the compositions described herein. Photodynamic therapy is a minimally invasive two-step medical procedure that uses photoactivatable drugs called photosensitizers to treat a range of diseases. First, a photosensitizer is administered and, once it has permeated the target tissue, the photosensitizer is then activated by exposure to a dose of electromagnetic (usually light) radiation at a particular wavelength. The compositions disclosed herein may contain a photosensitizer. In embodiments, any suitable photosensitizing agent or mixture of agents may be used herein. Generally, these will absorb radiation in the range of from about 380 nm to about 900 nm. As used herein, “photosensitizer” or “photosensitizing agent” preferably means a chemical compound which, when contacted by radiation of a certain wavelength, forms singlet oxygen or thermal energy. Non-limiting examples of photosensitizers include aminolevulinic acid esters, porphyrins, porphyrin derivatives, bacteriochlorins, isobacteriochlorins, phthalocyanine, naphthalocyanines, pyropheophorbides, sapphyrins, texaphyrins, tetrahydrochlorins, purpurins, porphycenes, phenothiaziniums, and metal complexes such as, but not limited to, tin, aluminum, zinc, lutetium, and tin ethyl etiopurpurin (SnET2), and combinations thereof.

The compositions of the present invention can also be administered in combination with other active ingredients, or other compatible drugs or compounds where such combination is seen to be desirable or advantageous in achieving the desired effects of the methods described herein.

This invention and embodiments illustrating the method and materials used may be further understood by reference to the following non-limiting examples.

Presented below are examples discussing generation of ECM compositions contemplated for the discussed applications. The following examples are provided to further illustrate the embodiments of the present invention, but are not intended to limit the scope of the invention. While they are typical of those that might be used, other procedures, methodologies, or techniques known to those skilled in the art may alternatively be used.

### EXAMPLES

### Example 1

An exemplary anhydrous composition is described below:

| Component | wt % | Role |
|---|---|---|
| tetrahydrofurfurylalcohol polyethylene | 38.55% | solvent |
| glycol ether | | |
| propylene glycol monocaprylate | 20% | co-solvent |
| Propylene Glycol | 10% | solvent |
| Poloxamer 407 | 10% | surfactant |
| erlotinib free base | 1.2% | API |
| cholesterol | 3% | emollient |
| Silicones | 15% | emollient |
| Klucel ® | 2.25% | gelling agent |

### Example 2

An exemplary anhydrous composition is described below:

| Component | wt % | Role |
|---|---|---|
| tetrahydrofurfurylalcohol | 28.47% | drug solvent |
| polyethylene glycol ether | | |
| PEG400 | 41% | co-solvent |
| Propylene Glycol | 10% | skin penetration |
| | | enhancer/solvent |
| erlotinib free base | 2.4% | API |
| Cholesterol | 0.90% | emollient |
| Cyclomethicone | 10% | emollient |
| Dimethicone | 5% | emollient |
| Klucel ® | 2.23% | gelling agent |

### Example 3

An exemplary anhydrous composition is described below:

| Component | wt % | Role |
|---|---|---|
| Capryilic/Capric | 32.5% | drug solvent |
| Triclycerides | | |
| Glycofural | 25% | co-solvent |
| Propylene Glycol | 10% | skin penetration |
| | | enhancer/solvent |
| Isopropyl myristate | 12% | emollient/thickening agent |
| erlotinib free base | 2.2% | API |
| Ascorbyl Palmitate | 0.3% | antioxidant |
| Cyclomethicone | 10% | emollient |
| Dimethicone | 5% | emollient |
| Carbopol | 3% | gelling agent |

### Example 4

An exemplary anhydrous composition is described below:

| component | Wt % | Role |
|---|---|---|
| Erlotinib free base | 3.26 | API |
| Isopropyl alcohol | 15 | solvent |
| PEG400 | 55.668 | solvent |
| Propylene glycol | 15 | solvent |
| Glycerol | 10 | solvent |
| Klucel ® | 1 | gelling agent |
| Propyl gallate | 0.05 | antioxidant |
| Ascorbyl palmitate | 0.02 | antioxidant |
| a-tocopherol | 0.002 | antioxidant |

### Example 5

An exemplary anhydrous composition is described below:

| component | Wt % | Role |
|---|---|---|
| Erlotinib free base | 3.26 | API |
| Isopropyl alcohol | 15 | solvent |
| PEG400 | 55.388 | solvent |
| Propylene glycol | 15 | solvent |
| Glycerol | 10 | solvent |
| Klucel ® | 1 | gelling agent |
| Propyl gallate | 0.05 | antioxidant |
| Ascorbyl palmitate | 0.02 | antioxidant |
| a-tocopherol | 0.002 | antioxidant |

### Example 6

An exemplary anhydrous composition is described below:

| component | Wt % | Role |
|---|---|---|
| Erlotinib free base | 4.54 | API |
| Isopropyl alcohol | 15 | solvent |
| PEG400 | 44.388 | solvent |
| Propylene glycol | 25 | solvent |
| Glycerol | 10 | solvent |
| Klucel ® | 1 | gelling agent |
| Propyl gallate | 0.05 | antioxidant |
| Ascorbyl palmitate | 0.02 | antioxidant |
| a-tocopherol | 0.002 | antioxidant |

### Example 7

An exemplary anhydrous composition is described below:

| component | Wt % | Role |
|---|---|---|
| Erlotinib free base | 4.0 | API |
| Isopropyl alcohol | 15 | solvent |
| PEG400 | 55.4 | solvent |
| diisopropyl adipate | 15 | solvent |
| Glycerol | 10 | solvent |
| Klucel ® | 0.75 | gelling agent |
| Propyl gallate | 0.05 | antioxidant |
| Ascorbyl palmitate | 0.02 | antioxidant |
| a-tocopherol | 0.002 | antioxidant |

### Example 8

An exemplary anhydrous composition is described below:

| component | Wt % | Role |
|---|---|---|
| Erlotinib free base | 4.51 | API |
| ethanol | 15 | solvent |
| PEG400 | 54.418 | solvent |
| diisopropyl adipate | 15 | solvent |
| Glycerol | 10 | solvent |
| Klucel ® | 1 | gelling agent |
| Propyl gallate | 0.05 | antioxidant |
| Ascorbyl palmitate | 0.02 | antioxidant |
| a-tocopherol | 0.002 | antioxidant |

### Example 9

An exemplary anhydrous composition is described below:

| component | Wt % | Role |
|---|---|---|
| Erlotinib free base | 2.384 | API |
| Isopropyl alcohol | 15 | solvent |
| PEG400 | 56.548 | solvent |
| Propylene glycol | 15 | solvent |
| Glycerol | 10 | solvent |
| Klucel ® | 1 | gelling agent |
| Propyl gallate | 0.05 | antioxidant |
| Ascorbyl palmitate | 0.02 | antioxidant |
| a-tocopherol | 0.002 | antioxidant |

### Example 10

An exemplary anhydrous composition is described below:

| component | Wt % | Role |
|---|---|---|
| Erlotinib free base | 2.69 | API |
| Isopropyl alcohol | 15 | solvent |
| PEG400 | 26.238 | solvent |
| Propylene glycol | 15 | penetration |
| | | enhancer/solvent |
| Transuctol P | 25 | penetration |
| | | enhancer/solvent |
| Diisopropyl adipate | 15 | solvent |
| Klucel ® | 1 | gelling agent |
| Propyl gallate | 0.05 | antioxidant |
| Ascorbyl palmitate | 0.02 | antioxidant |

### Example 11

An exemplary anhydrous composition is described below:

| component | Wt % | Role |
|---|---|---|
| Erlotinib free base | 3.254 | API |
| Isopropyl alcohol | 15 | solvent |
| PEG400 | 47.608 | solvent |
| Propylene glycol | 15 | Penetration |
| | | enhancer/solvent |
| Transcutol P | 10 | penetration |
| | | enhancer/solvent |
| Glycerol | 10 | solvent |
| Klucel ® | 1 | gelling agent |
| Propyl gallate | 0.05 | antioxidant |
| Ascorbyl palmitate | 0.02 | antioxidant |
| a-tocopherol | 0.002 | antioxidant |

### Example 12

An exemplary ointment composition is described below:

| component | Wt % | Role |
|---|---|---|
| Erlotinib free base | 4.59 | API |
| PEG400 | 34.34 | solvent |
| PEG 3350 | 13 | solvent |
| Transcutol P | 47.998 | penetration |
| | | enhancer/solvent |
| Propyl gallate | 0.05 | antioxidant |
| Ascorbyl palmitate | 0.02 | antioxidant |
| a-tocopherol | 0.002 | antioxidant |

### Example 13

An exemplary anhydrous composition is described below:

| component | Wt % | Role |
|---|---|---|
| Erlotinib free base | 3.9 | API |
| Isopropyl alcohol | 15 | solvent |
| PEG400 | 51 | solvent |
| Propylene glycol | 1.5 | penetration |
| | | enhancer/solvent |
| Diisopropyl adipate | 15 | solvent |
| Glycerol | 10 | solvent |
| Benzyl alcohol | 2 | solvent |
| Oleyl alcohol | 0.75 | solvent |
| Klucel ® | 0.75 | gelling agent |
| Propyl gallate | 0.05 | antioxidant |
| BHT | 0.02 | antioxidant |
| a-tocopherol | 0.002 | antioxidant |

### Example 14

An exemplary anhydrous composition is described below:

| component | Wt % | Role |
|---|---|---|
| Erlotinib free base | 3.9 | API |
| PEG400 | 28.3 | solvent |
| Propylene glycol | 15 | Penetration |
| | | enhancer/solvent |
| Transcutol P | 15 | penetration |
| | | enhancer/solvent |
| Diisopropyl adipate | 15 | solvent |
| Glycerol | 10 | solvent |
| Benzyl alcohol | 2 | solvent |
| Oleyl alcohol | 10 | solvent |
| Klucel ® | 0.75 | gelling agent |
| Propyl gallate | 0.05 | antioxidant |
| BHT | 0.02 | antioxidant |
| a-tocopherol | 0.002 | antioxidant |

### Example 15

An exemplary anhydrous composition is described below:

| component | Wt % | Role |
|---|---|---|
| Erlotinib free base | 3.2 | API |
| PEG400 | 51.7 | solvent |
| Propylene glycol | 1.5 | penetration |
| | | enhancer/solvent |
| Transcutol | 15 | penetration |
| | | enhancer/solvent |
| Diisopropyl adipate | 15 | solvent |
| Glycerol | 10 | solvent |
| Benzyl alcohol | 2 | solvent |
| Oleyl alcohol | 0.75 | solvent |
| Klucel ® | 0.75 | gelling agent |
| Propyl gallate | 0.05 | antioxidant |
| Ascorbyl palmitate | 0.02 | antioxidant |
| a-tocopherol | 0.002 | antioxidant |

### Example 16

An exemplary anhydrous composition is described below:

| component | Wt % | Role |
|---|---|---|
| Erlotinib free base | 4 | API |
| PEG400 | 55.25 | solvent |
| Diisopropyl adipate | 15 | solvent |
| Glycerin | 10 | solvent |
| IPA | 15 | solvent |
| hydroxypropyl | 0.75 | Gelling agent |
| cellulose | | |

### Example 17

An exemplary anhydrous composition is described below:

| component | Wt % | Role |
|---|---|---|
| Erlotinib free base | 4 | API |
| PEG400 | 40.25 | solvent |
| Diisopropyl adipate | 20 | solvent |
| Glycerin | 20 | solvent |
| IPA | 15 | solvent |
| hydroxypropyl | 0.75 | Gelling agent |
| cellulose | | |

### Example 18

An exemplary anhydrous composition is described below:

| component | Wt % | Role |
|---|---|---|
| Erlotinib free base | 1 | API |
| PEG400 | 40.25 | solvent |
| Diisopropyl adipate | 20 | solvent |
| Glycerin | 20 | solvent |
| IPA | 18 | solvent |
| hydroxypropyl | 0.75 | Gelling agent |
| cellulose | | |

### Example 19

An exemplary anhydrous composition is described below:

| component | Wt % | Role |
|---|---|---|
| Erlotinib free base | 0.3 | API |
| PEG400 | 38.95 | solvent |
| Diisopropyl adipate | 20 | solvent |
| Glycerin | 20 | solvent |
| IPA | 20 | solvent |
| hydroxypropyl | 0.75 | Gelling agent |
| cellulose | | |

### Example 20

An exemplary anhydrous composition is described below:

| component | Wt % | Role |
|---|---|---|
| Erlotinib free base | 1.0 | API |
| Isopropyl alcohol | 10.0 | solvent |
| PEG400 | 62.9 | solvent |
| Diisopropyl adipate | 15.0 | solvent |
| Glycerin | 10.0 | solvent |
| Klucel ® | 1.0 | gelling agent |
| BHT | 0.1 | antioxidant |

### Example 21

An exemplary anhydrous composition is described below:

| component | Wt % | Role |
|---|---|---|
| Erlotinib free base | 1.0 | API |
| Isopropyl alcohol | 10.0 | solvent |
| PEG400 | 62.9 | solvent |
| Diisopropyl adipate | 15.0 | solvent |
| Glycerol | 10.0 | solvent |
| Klucel ® | 1.0 | gelling agent |
| BHT | 0.1 | antioxidant |

### Example 22

An exemplary anhydrous composition is described below:

| component | Wt % | Role |
|---|---|---|
| Erlotinib free base | 3.0 | API |
| Isopropyl alcohol | 10.0 | solvent |
| PEG400 | 60.9 | solvent |
| Diisopropyl adipate | 15.0 | solvent |
| Glycerol | 10.0 | solvent |
| Klucel ® | 1.0 | gelling agent |
| BHT | 0.1 | antioxidant |

### Example 23

An exemplary anhydrous composition is described below:

| component | Wt % | Role |
|---|---|---|
| Erlotinib free base | 4.0 | API |
| Isopropyl alcohol | 10.0 | solvent |
| PEG400 | 59.9 | solvent |
| Diisopropyl adipate | 15.0 | solvent |
| Glycerol | 10.0 | solvent |
| Klucel ® | 1.0 | gelling agent |
| BHT | 0.1 | antioxidant |

### Example 24: Evaluating Topical Bioavailability

Dermatopharmacokinetic (DPK) Studies—The dermatopharmacokinetic (DPK) approach is comparable to a blood, plasma, urine PK approach applied to the stratum corneum. DPK encompasses drug concentration measurements with respect to time and provides information on drug uptake, apparent steady-state levels, and drug elimination from the stratum corneum based on a stratum corneum concentration-time curve.

Application and Removal of Test and Reference Products: The treatment areas will be marked using a template without disturbing or injuring the stratum corneum/skin. The size of the treatment area will depend on multiple factors including drug strength, analytical sensitivity, the extent of drug diffusion, and exposure time. The stratum corneum is highly sensitive to certain environmental factors. To avoid bias and to remain within the limits of experimental convenience and accuracy, the treatment sites and arms will be randomized. Uptake, steady-state, and elimination phases, as described in more detail below, may be randomized between the right and left arms in a subject. Exposure time points in each phase may be randomized among various sites on each arm. The test and reference products for a particular exposure time point may be applied on sites to minimize differences. Test and reference products should be applied concurrently on the same subjects according to a SOP that has been previously developed and validated. The premarked sites will be treated with predetermined amounts of the products (e.g., 5 mg/sq cm) and covered with a nonocclusive guard. Occlusion will be used only if recommended in product labeling. Removal of the drug product will be performed according to SOPs at the designated time points, using multiple cotton swabs or Q-tips with care to avoid stratum corneum damage. In case of certain oily preparations such as ointments, washing the area with a mild soap may be needed before skin stripping. If washing is carried out, it will be part of an SOP.

Sites and Duration of Application: The bioavailability/bioequivalence (BA/BE) study will include measurements of drug uptake into the stratum corneum and drug elimination from skin. A minimum of eight sites will be employed to assess uptake/elimination from each product. The time to reach steady state in the stratum corneum will be used to determine timing of samples. For example, if the drug reaches steady-state in three hours, 0.25, 0.5, 1 and 3 hours posttreatment may be selected to determine uptake and 4, 6, 8 and 24 hours may be used to assess elimination. A zero time point (control site away from test sites) on each subject will be selected to provide baseline data. If the test/reference drug products are studied on both forearms, randomly selected sites on one arm may be designated to measure drug uptake/steady-state. Sites on the contralateral arm may then be designated to measure drug elimination. During drug uptake, both the excess drug removal and stratum corneum stripping times are the same so that the stratum corneum stripping immediately follows the removal of the excess drug. In the elimination phase, the excess drug will be removed from the sites at the steady-state time point, and the stratum corneum will be harvested at succeeding times over 24 hours to provide an estimate of an elimination phase.

Collection of Sample: Skin stripping proceeds first with the removal of the first 1-2 layers of stratum corneum with two adhesive tapes strip/disc applications, using a commercially available product (e.g., D-Squame, Transpore). These first two tape-strip(s) contain the generally unabsorbed, as opposed to penetrated or absorbed, drug and therefore will be analyzed separately from the rest of the tape-strips. The remaining stratum corneum layers from each site will be stripped at the designated time intervals. This is achieved by stripping the site with an additional 10 adhesive tape-strips. All ten tape strips obtained from a given time point will be combined and extracted, with drug content determined using a validated analytical method. The values will be generally expressed as amounts/area (e.g., ng/cm2) to maintain uniformity in reported values. Data may be computed to obtain full drug concentration-time profiles, Cmax-ss, Tmax-ss, and AUCs for the test and reference products.

Procedure for Skin Stripping—To assess drug uptake: The test and/or reference drug products will be applied concurrently at multiple sites. After an appropriate interval, the excess drug from a specific site will be removed by wiping three times lightly with a tissue or cotton swab. Using information from the pilot study, the appropriate times of sample collection to assess drug uptake will be determined. The application of adhesive tape two times will be repeated, using uniform pressure, discarding these first two tape strips. Stripping will be continued at the same site to collect ten more stratum corneum samples. Care will be taken to avoid contamination with other sites. The procedure will be repeated for each site at other designated time points. The drug will be extracted from the combined ten skin strippings and the concentration will be determined using a validated analytical method. The results will be expressed as amount of drug per square cm treatment area of the adhesive tape.

To assess drug elimination: The test and reference drug product will be applied concurrently at multiple sites chosen based on the results of the pilot study. Sufficient exposure period to reach apparent steady-state level will be allowed. Excess drug from the skin surface will be removed as described previously, including the first two skin strippings. The skin stripping samples will be collected using ten successive tape strips at time intervals based on the pilot study and drug content will be analyzed.

Metrics and Statistical Analyses: A plot of stratum corneum drug concentration versus a time profile will be constructed to yield stratum corneum metrics of Cmax, Tmax and AUC. The two one-sided hypotheses at the a=0.05 level of significance will be tested for AUC and Cmax by constructing the 90 percent confidence interval (CI) for the ratio between the test and reference averages. Individual subject parameters, as well as summary statistics (average, standard deviation, coefficient of variation, 90% CI) will be reported. For the test product to be BE, the 90 percent CI for the ratio of means (population geometric means based on log-transformed data) of test and reference treatments will fall within 80-125 percent for AUC and 70-143 percent for Cmax.

In vivo Dermal Open Flow Microperfusion: In dermal open-flow microperfusion (dOFM), a thin, hollow tube will be inserted just under the skin surface, running through a section of the skin a few inches wide and then exiting. A liquid similar to body fluid will be injected into the tubing; a portion of the tube under the skin is porous, so any drug that has been applied and absorbed through the skin's outer layer enters the flowing liquid, which will be then collected for analysis. dOFM can reliably measure the changing amounts of drug in the skin after topical application of a dermatological drug product.

### Example 25: Effect of Erlotinib on 2D Non-Diseased Keratinocytes

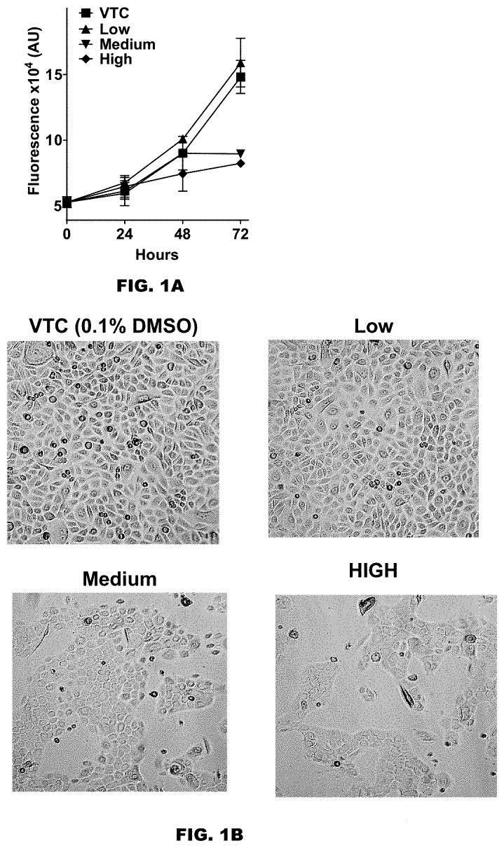

Normal keratinocyte cells were cultured and treated with vehicle or erlotinib at 100, 500 and 1000 nM. The proliferation rate of the cells was determined. Medium and high doses of erlotinib decreased keratinocyte proliferation (see FIGS. 1A-B). High dose erlotinib induced increased Loricrin expression. This data shows that keratinocyte differentiation is increased following exposure to erlotinib

### Example 26: Effect of Erlotinib on 3D Human Skin Equivalent

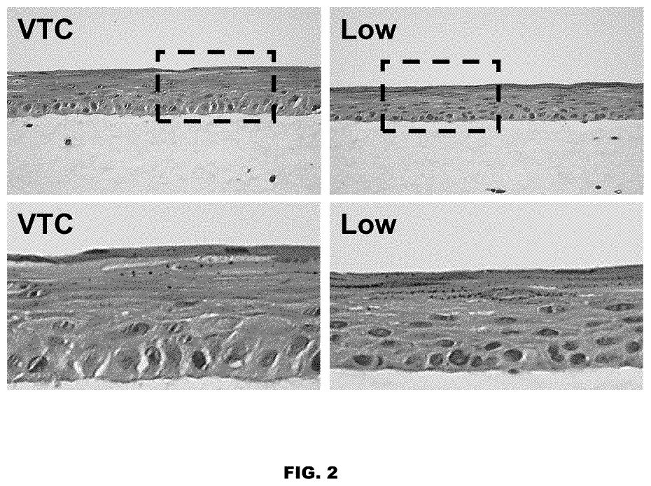

Human skin equivalents were prepared by methods known in the art. Examples of such methods are disclosed in Mieremet et al. Int. J. Mol. Sci. (2021) 22(11):5790. Non-diseased cells were plated for 3D human skin equivalent (HSE) and treated with 100 nM erlotinib from the time of lifting to the air-liquid interface every 72 hours until harvest at day 10. The samples were subjected to protein, mRNA, immunofluorescence and morphological analysis. FIG. 2 shows that 100 nM erlotinib enhances terminal differentiation. Additionally, it was observed that the desmosomal organization is enhanced.

### Example 27: Effect of Erlotinib on 2D Cultured Keratinocytes Derived from Non-Diseased and Epidermolytic Ichthyosis

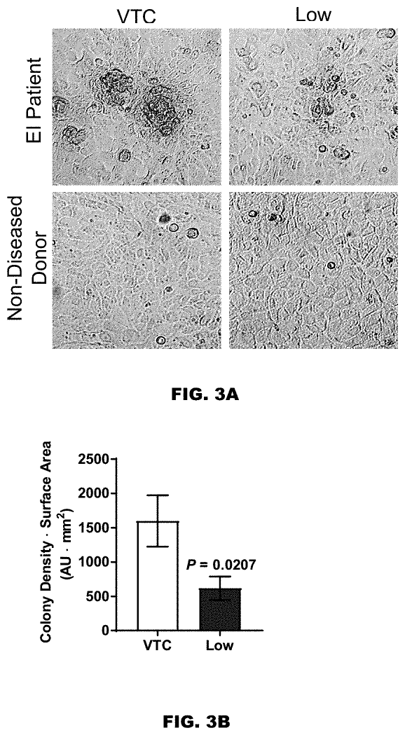

It has been noted that epidermolytic ichthyosis (EI) patient samples retain a disease-like phenotype in 2D cultures. Tissue from non-diseased donors and EI patients were cultured and treated with 100 nM erlotinib. Following exposure to 100 nM erlotinib EI derived keratinocyte colony density is markedly reduced as observed under the microscope (FIG. 3A) and by fold change (FIG. 3B).

### Example 28: Effect of Erlotinib on 3D Non-Diseased and EI 3D HSE

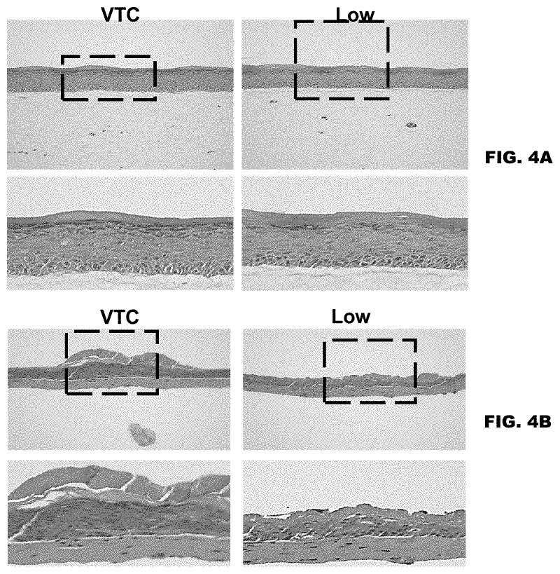

Non-diseased cells and cells from an EI patient were plated for 3D human skim equivalent (HSE)and treated with 100 nM erlotinib from the time of lifting to the air-liquid interface every 72 hours until harvest at day 12. The samples were subjected to morphological analysis. A marked morphologic change is produced in the upper epidermis in erlotinib treated EI constructs suggesting that aberrant proliferation and differentiation in the disease can be decreased by this agent. (FIGS. 4A-B).

### Example 29: Effect of Erlotinib on Epidermal Barrier Function

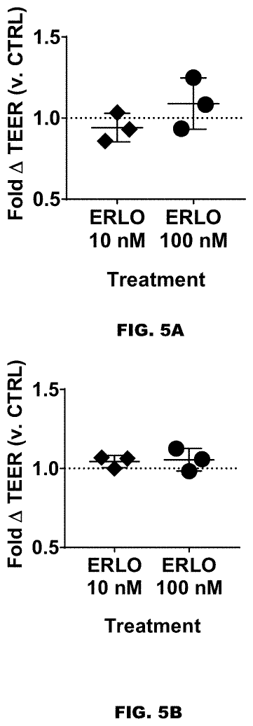

Transepithelial electrical resistance (TEER) can be used to measure epidermal barrier function (a combination of the lipid and tight junction barriers) in 3D epidermal cultures._Non-diseased cells and cells from an EI patient were plated for 3D human skim equivalent (HSE)and treated with the erlotinib at 10 nM and 100 nM from the time of lifting to the air-liquid interface every 72 hours until harvest at day 10 The results indicate that erlotinib does not impair epidermal barrier function in non-diseased tissue cultures or tissue cultures from EI patients (FIGS. 5A-B).

### Example 30: Effect of Erlotinib on Gene Expression on 3D Non-Diseased and EI 3D HSE

Non-diseased cells and cells from an EI patient were plated for 3D human skin equivalent (HSE)and treated with the erlotinib at 10 nM or 100 nM from the time of lifting to the air-liquid interface every 72 hours until harvest at day 10. The samples were subjected to gene expression analysis. The results are shown in the Tables below. The results indicate that treatment of normal and EI derived human keratinocytes in 3D culture with erlotinib resulted marked increase in keratin 10 (KRT10) expression. Markers of terminal differentiation such as fillaggrin (FLG) and loricrin (LOR) and markers of cell adhesion such as desmoglein 1 (DSG 1) were also modified.

Gene expression results for non-diseased tissue following treatment with erlotinib:

| | NUTC394.14 | | | | | | | | | | |
|---|---|---|---|---|---|---|---|---|---|---|---|
| Treatment | FLG | LOR | IVL | KRT10 | KRT16 | DSG1 | DSC1 | CDN4 | CLDN1 | TJP1 | OCLN |
| Control | 1.00 | 1.00 | 1.00 | 1.00 | 1.00 | 1.00 | 1.00 | 1.00 | 1.00 | 1.00 | 1.00 |
| Erlo 10 nM | 0.50 | 0.03 | 2.11 | 0.27 | 1.67 | 0.72 | 0.59 | 2.87 | 1.28 | 1.62 | 1.73 |
| Erlo 100 nM | 2.21 | 7.66 | 1.14 | 10.75 | 0.89 | 2.59 | 2.52 | 1.54 | 1.16 | 1.48 | 1.27 |
| | NHEK83.19 | | | | | | | | | | |
| Treatment | FLG | LOR | IVL | KRT10 | KRT16 | DSG1 | DSC1 | CDN4 | CLDN1 | TJP1 | OCLN |
| Control | 1.00 | 1.00 | 1.00 | 1.00 | 1.00 | 1.00 | 1.00 | 1.00 | 1.00 | 1.00 | 1.00 |
| Erlo 10 nM | 0.51 | 0.03 | 2.15 | 0.28 | 1.70 | 0.73 | 0.60 | 2.92 | 1.30 | 1.65 | 1.76 |
| Erlo 100 nM | 2.37 | 8.19 | 1.21 | 11.50 | 1.00 | 2.77 | 2.69 | 1.65 | 1.24 | 1.58 | 1.36 |
| | NUTC439.14 | | | | | | | | | | |
| Treatment | FLG | LOR | IVL | KRT10 | KRT16 | DSG1 | DSC1 | CDN4 | CLDN1 | TJP1 | OCLN |
| Control | 1.0 | 1.0 | 1.0 | 1.0 | 1.0 | 1.0 | 1.0 | 1.0 | 1.0 | 1.0 | 1.0 |
| Erlo 10 nM | 1.0 | 1.4 | 0.9 | 2.1 | 0.9 | 2.1 | 1.9 | 0.7 | 1.9 | 0.7 | 0.7 |
| Erlo 100 nM | 25.9 | — | 1.5 | 17.2 | 1.6 | 9.3 | 29.4 | 1.4 | 29.4 | 1.3 | 1.6 |

Gene expression results for EI patient tissue following treatment with erlotinib:

| | CMH101.11 | | | | | | | | | | |
|---|---|---|---|---|---|---|---|---|---|---|---|
| Treatment | FLG | LOR | IVL | KRT10 | KRT16 | DSG1 | DSC1 | CDN4 | CLDN1 | TJP1 | OCLN |
| Control | 1.00 | 1.00 | 1.00 | 1.00 | 1.00 | 1.00 | 1.00 | 1.00 | 1.00 | 1.00 | 1.00 |
| Erlo 10 nM | 1.08 | 1.10 | 1.04 | 1.00 | 1.63 | 1.11 | 0.65 | 0.96 | 0.71 | 0.55 | 0.81 |
| Erlo 100 nM | 0.72 | 0.95 | 0.83 | 5.78 | 0.85 | 2.57 | 6.09 | 0.72 | 0.37 | 0.57 | 0.57 |
| | CMH102.11 | | | | | | | | | | |
| Treatment | FLG | LOR | IVL | KRT10 | KRT16 | DSG1 | DSC1 | CDN4 | CLDN1 | TJP1 | OCLN |
| Control | 1.00 | 1.00 | 1.00 | 1.00 | 1.00 | 1.00 | 1.00 | 1.00 | 1.00 | 1.00 | 1.00 |
| Erlo 10 nM | 1.22 | 0.90 | 1.58 | 2.27 | 1.31 | 4.72 | 0.79 | 1.86 | 0.79 | 1.13 | 1.15 |
| Erlo 100 nM | 2.06 | 0.79 | 1.76 | 6.44 | 1.14 | 6.61 | 2.20 | 3.17 | 2.20 | 1.44 | 1.92 |
| | CMH98.11 | | | | | | | | | | |
| Treatment | FLG | LOR | IVL | KRT10 | KRT16 | DSG1 | DSC1 | CDN4 | CLDN1 | TJP1 | OCLN |
| Control | 1.00 | 1.00 | 1.00 | 1.00 | 1.00 | 1.00 | 1.00 | 1.00 | 1.00 | 1.00 | 1.00 |
| Erlo 10 nM | 1.2 | 1.2 | 1.3 | 0.9 | 1.3 | 1.8 | 0.8 | 0.7 | 0.9 | 0.7 | 0.7 |
| Erlo 100 nM | 1.1 | 0.6 | 2.3 | 1.6 | 0.8 | 1.9 | 1.7 | 1.8 | 1.6 | 1.2 | 1.2 |

### Example 31: Effect of Erlotinib in a Mouse Model of Hyperkeratosis

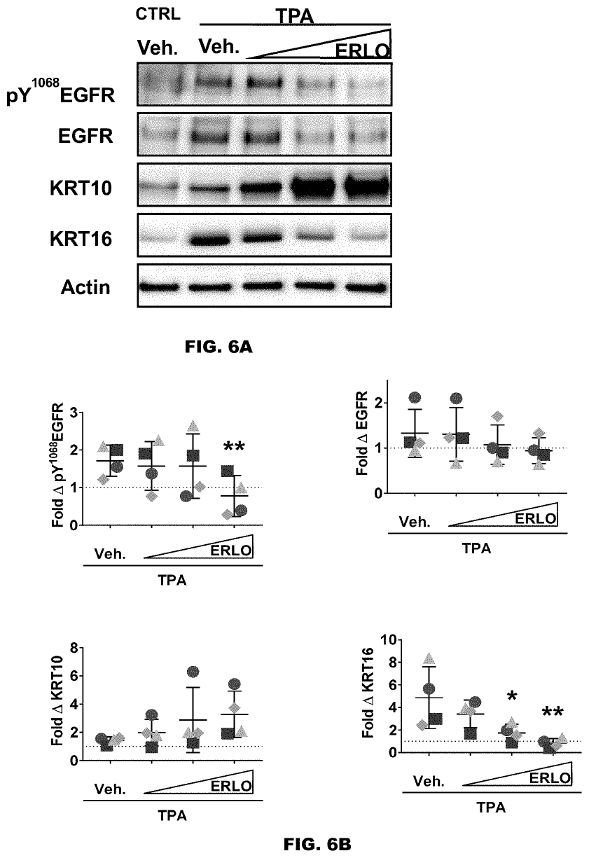

Hairless Skh-1 mice were treated with 12-O-Tetradecanoylphorbol-13-acetate (TPA) daily for 7 days than every other day for the next 7 days to induce skin inflammation. The mice were then treated with vehicle or a composition comprising 0.4%, 1.0% or 4.0% erlotinib daily starting on Day 8. The mice were assessed for degrees of inflammation, transepidermal water loss and expression of p^{Y1068}EGFR, EGFR, KRT10 and KRT16. Treatment with erlotinib resulted dose dependent reduction of skin inflammation as well as significantly decreasing transepithelial water loss. Further, treatment with erlotinib resulted in dose dependent change in the expression of p^{Y1068}EGFR, EGFR, KRT10 and KRT16 (see FIGS. 6A-B).

Although the invention has been described with reference to the above examples, it will be understood that modifications and variations are encompassed within the spirit and scope of the invention. Accordingly, the invention is limited only by the following claims.

1. A topical anhydrous composition of an EGFR inhibitor comprising:
one or more EGFR inhibitors or pharmaceutically acceptable salts, hydrates, solvates, polymorphs, or amorphous solid thereof in an amount of about 0.1 wt % to about 20 wt % of the topical anhydrous composition,
one or more solvents in an amount of about 75 wt % to about 99.9 wt % of the topical anhydrous composition, and
one or more gelling agents in an amount of about 0.1 wt % to about 6 wt % of the topical anhydrous composition.

2. The topical anhydrous composition of claim 1, further comprising one or more antioxidants, wherein the one or more antioxidants comprises about 0.01 wt % to about 1 wt % of the topical anhydrous composition.

3. The topical anhydrous composition of claim 1, wherein:
(i) the one or more EGFR inhibitor comprises about 0.1 wt % to about 7 wt % of the topical anhydrous composition;
(ii) the one or more solvent comprises about 85 wt % to about 98 wt % of the topical anhydrous composition; and
(iii) the one or more gelling agent comprises about 0.1 wt % to about 1 wt % of the topical anhydrous composition.

4. The topical anhydrous composition of claim 1, wherein the one or more EGFR inhibitor is the free base.

5. (canceled)

6. (canceled)

7. (canceled)

8. The topical anhydrous composition of claim 1, wherein the EGFR inhibitor is selected from the group consisting of erlotinib, osimertinib (AZD9291), neratinib, gefitinib, dacomitinib, lapatinib, vandetanib, afatinib, icotinib, rociletinib, naquotinib (ASP8273), and pharmaceutically acceptable salts, hydrates, solvates, polymorphs, or amorphous solids thereof.

9. The topical anhydrous composition of claim 1, further comprise monoclonal antibodies selected from panitumumab, nimotuzumab, necitumumab, LY3016859, or cetuximab.

10. The topical anhydrous composition of claim 1, wherein the one or more solvent is selected from alcohols, polyols, amides, esters, propylene glycol ethers, or mixtures thereof.

11. The topical anhydrous composition of claim 10, wherein the alcohol or polyol is selected from ethanol, isopropanol, butanol, benzyl alcohol, ethylene glycol, propylene glycol, propylene glycol monocaprylate, diglycol, diethylene glycol monoethylether, tetrahydrofurfurylalcohol polyethylene glycol ether (glycofurol), butylene glycol, diethylene glycol, triethylene glycol, PEG 400, PEG 3350, SR-PEG 400, SR-DMI, oleyl alcohol, castor oil, miglyol 810, liquid paraffin, propylene glycol dicaprylate/dicaprate, butanediols, isomers of butanediols, glycerol (AKA glycerin), glycerol triacetate, pentaerythritol, sorbitol, mannitol, diisopropyl adipate, dimethyl isosorbide, polyethylene glycol, polypropylene glycol, polyvinylalcohol, hydroxypropyl methylcellulose, cellulose derivatives, cyclodextrins and cyclodextrin derivatives, or mixtures thereof.

12. The topical anhydrous composition of claim 10, wherein the amide is selected from 2-pyrrolidone, 2-piperidone, c-caprolactam, N-alkylpyrrolidone, N-hydroxyalkylpyrrolidone, N-alkylpiperidone, N-alkylcaprolactam, dimethylacetamide, polyvinylpyrrolidone, or mixtures thereof.

13. The topical anhydrous composition of claim 1, wherein the one or more solvents are selected from PEG400, diisopropyl adipate, glycerol, isopropyl alcohol, or mixtures thereof.

14. The topical anhydrous composition of claim 10, wherein the ester is selected from ethyl propionate, tributylcitrate, acetyl triethylcitrate, acetyl tributyl citrate, triethylcitrate, ethyl oleate, ethyl caprylate, ethyl butyrate, triacetin, propylene glycol monoacetate, propylene glycol diacetate, c-caprolactone, isomers of c-caprolactone, 6-valerolactone, isomers of 6-valerolactone, P-butyrolactone, isomers of P-butyrolactone, or mixtures thereof.

15. The topical anhydrous composition of claim 1, wherein the one or more gelling agents are selected from poloxamers, carbomers, or mixtures thereof.

16. The topical anhydrous composition of claim 15, wherein the poloxamer is selected from poloxamer P-188, poloxamer P-138, poloxamer P-237, poloxamer P-288, poloxamer P-124, poloxamer P-338, poloxamer P-407, poly(ethylene glycol/DL-lactide-Co-glyceride) poly(caprolactam), hydroxypropyl cellulose (HPC), glyceryl tris 12-hydroxy stearate, hydroxy stearin, propylene carbonate, polyvinyl pyrolidone, or mixtures thereof.

17. The topical anhydrous composition of claim 15, wherein the carbomer is selected from carbomer 981, carbomer 934, carbomer 934P, carbomer 940, carbomer 941, carbomer 1342, polycarbophil, calcium polycarbophil, or mixtures thereof.

18. The topical anhydrous composition of claim 1, wherein the one or more gelling agent is hydroxypropyl cellulose (HPC).

19. The topical anhydrous composition of claim 2, wherein the one or more antioxidants are selected from ascorbic acid, vitamin E and its derivatives, a-tocopherol, y-tocopherol, 5-tocopherol, ascorbyl palmitate, propyl gallate (PG), octyl gallate, dodecyl gallate, butylated hydroxy anisole (BHA), butylated hydroxy toluene (BHT), D-a-tocopheryl polyethylene glycol 1000 succinate, or mixtures thereof. Preferably, the one or more antioxidants is selected from propyl gallate, ascorbyl palmitate, or mixtures thereof.

20. The topical anhydrous composition of claim 2, wherein the one or more antioxidants are selected from the group consisting of butylated hydroxy toluene (BHT), ascorbyl palmitate, propyl gallate, a-tocopherol, or mixtures thereof.

21. A method of treating a skin disorder in a subject in need thereof comprising topically administering an effective amount of a topical anhydrous composition of an EGFR inhibitor comprising:
one or more EGFR inhibitors or pharmaceutically acceptable salts, hydrates, solvates, polymorphs, or amorphous solid thereof in an amount of about 0.1 wt % to about 20 wt % of the topical anhydrous composition,
one or more solvents in an amount of about 75 wt % to about 99.9 wt % of the topical anhydrous composition, and
one or more gelling agents in an amount of about 0.1 wt % to about 6 wt % of the topical anhydrous composition.

22. The method of claim 21, wherein the skin disorder selected from plantar hyperkeratosis, blisters, tuberous sclerosis, seborrheic keratosis, keratosis pilaris, epidermolysis bullosa, multiple minute digitate hyperkeratosis, hyperkeratosis lenticularis perstans, stasis dermatitis, focal acral hyperkeratosis, follicular hyperkeratosis, lichenoid keratoses (lichen planus, lichen sclerosus), chronic erosive oral lichen, Conradi-Eltinermann, epidermolytic ichthyosis, erythrokeratoderma variabilis, ichthyosis hystrix, KID syndrome, Netherton syndrome, Olmsted syndrome, Refsum disease, Sj ogren-Larsson Syndrome, actinic keratosis, pachyonychia congenita, hyperhidrosis, warts, calluses, dermatitis (contact dermatitis, drug-induced dermatitis, allergic dermatitis, nummular dermatitis, perioral dermatitis, neurodermatitis, seborrheic dermatitis, and atopic dermatitis), psoriasis, acne, carbunculosis, cellulitis, furunculosis, granuloma, acanthosis nigricans, athlete's foot, bacterial vaginosis, balanitis, dermatofibrosarcoma protruberans, basal cell carcinoma, squamous cell carcinoma, melanoma, merkel cell carcinoma, keloid, cystic lymphangioma, Cavernous lymphangioma, venous malformation, epidermal nevi, bromhidrosis, dermatophytosis, candidiasis, onychomycosis, tinea (tinea alba, tinea pedis, tinea unguium, tinea manuum, tinea cruris, tinea corporis, tinea capitis, tinea faciei, tinea barbae, tinea imbricata, tinea nigra, tinea versicolor, tinea incognito), eczema, dyshydrotic eczema, decubitous ulcer, ecthyma, erysipalus, erythema multiforme, impetigo, insect bites, genital warts, hemangioma, herpes, hives, hyperhidrosis, filariasis, lentigines, lupus, miliaria, milker's nodules, molluscum contagiosum, myiasis, scabies, cutaneous larva migrans, furuncular myiasis, migratory myiasis, pediculosis, nevus araneus, panniculitis, paronychia, pemphigoid, pityriasis, pruritis vulvae, rosacea, trichomoniasis, vaginal yeast infection, vitiligo, xeroderma, angiofibroma, Bannayan-Riley-Ruvalcaba syndrome, basal cell nevus syndrome, Birt-Hogg-Dube syndrome, Blue rubber bleb nevus syndrome, Cowden disease, cutaneous T-cell lymphoma, diffuse microcystic lymphatic malformations, epidermolysis bullosa simplex, extramammary paget, familial multiple discoid fibromas, Hailey-Hailey disease, infantile hemangiomas, juvenile polyposis syndrome, Kaposi sarcoma, Kaposiform hemangioendothelioma, Keloid scar disease, Lhermitte-Duclos syndrome, metastatic melanoma, Muir-Tone syndrome, neurofibromatosis, nonmelanoma skin cancer, oral graft-versus-host disease, Pemphigus vulgaris, Peutz-Jeghers syndrome, Port-wine stains, Proteus syndrome, Proteus-like Syndrome, refractory hemangioendotheliomas in Maffucci syndrome, Sturge-weber syndrome, hereditary footpad hyperkeratosis (HFH) in canines, cutaneous sarcoidosis, cutaneous Castleman Disease, Bullous Pemphigoid, Darier's disease (also known as keratosis follicularis or Darier-White disease), epidermolysis, ichthyosis, Lamellar Ichthyosis, and combinations thereof.

23-31. (canceled)

32. The method of claim 22, wherein the skin disorder is Hailey-Hailey Disease, Darier's disease, epidermolysis, epidermolysis bullosa simplex, Olmsted Syndrome, ichthyosis, epidermolytic ichthyosis, lamellar ichthyosis, acanthosis nigricans, or a combination thereof.
